SEQUENCE LISTING (22 sequences)

SEQ ID NO: 1 - protein, 308 aa, Homo sapiens
MQPSGHRLRDVEHHPLLAENDNYDSSSSSSSEADVADRVWFIRDGCGMICAVMTWLLVAYADFVVTFVMLLPSKDFWYSVVNGVIFNCLAVLALSSHLRTMLTDPGAVPKGNATKEYMESLQLKPGEVIYKCPKCCCIKPERAHHCSICKRCIRKMDHHCPWVNNCVGEKNQRFFVLFTMYIALSSVHALILCGFQFISCVRGQWTECSDFSPPITVILLIFLCLEGLLFFTFTAVMFGTQIHSICNDETEIERLKSEKPTWERRLRWEGMKSVFGGPPSLLWMNPFVGFRFRRLPTRPRKGGPEFSV

SEQ ID NO: 2 - protein, 308 aa, Mus musculus
MQPSGHRLRDIEHHPLLTDNDNYDSASSSSSETDMADRVWFIRDGCGMVCAVMTWLLVVYADFVVTFVMLLPSKDFWYSVVNGVLFNCLAVLALSSHLRTMLTDPGAVPKGNATKEYMESLQLKPGEVIYKCPKCCCIKPERAHHCSICKRCIRKMDHHCPWVNNCVGEKNQRFFVLFTMYIALSSVHALILCGLQFISCVRGQWTECSDFSPPITVILLVFLCLEGLLFFTFTAVMFGTQIHSICNDETEIERLKSEKPTWERRLRWEGMKSVFGGPPSLLWMNPFVGFRLRRLQMRTRKGGPEFSV

SEQ ID NO: 3 - protein, 299 aa, Homo sapiens
MMLIPTHHFRNIERKPEYLQPEKCVPPPYPGPVGTMWFIRDGCGIACAIVTWFLVLYAEFVVLFVMLIPSRDYVYSIINGIVFNLLAFLALASHCRAMLTDPGAVPKGNATKEFIESLQLKPGQVVYKCPKCCSIKPDRAHHCSVCKRCIRKMDHHCPWVNNCVGENNQKYFVLFTMYIALISLHALIMVGFHFLHCFEEDWTKCSSFSPPTTVILLILLCFEGLLFLIFTSVMFGTQVHSICTDETGIEQLKKEERRWAKKTKWMNMKAVFGHPFSLGWASPFATPDQGKADPYQYVV

SEQ ID NO: 4 - protein, 299 aa, Mus musculus
MMLIPTHHFRDIERKPEYLQPEKCAPPPFPGPAGAMWFIRDGCGIACAIVTWFLVLYAEFVVLFVMLVPSRDYAYSIINGIVFNLLAFLALASHCRAMLTDPGAVPKGNATKEFIESLQLKPGQVVYKCPKCCSIKPDRAHHCSVCKRCIRKMDHHCPWVNNCVGENNQKYFVLFTMYIALISLHALIMVGFHFLHCFEEDWTKCSSFSPPTTVILLILLCFEALLFLIFTSVMFGTQVHSICTDETGIEQLKKEERRWAKKTKWMNMKAVFGHPFSLGWASPFATPDQGKADPYQYVV

SEQ ID NO: 5 - protein, 231 aa, Homo sapiens
MCGNTMSVPLLTDAATVSGAERETAAVIFLHGLGDTGHSWADALSTIRLPHVKYICPHAPRIPVTLNMKMVMPSWFDLMGLSPDAPEDEAGIKKAAENIKALIEHEMKNGIPANRIVLGGFSQGGALSLYTALTCPHPLAGIVALSCWLPLHRAFPQAANGSAKDLAILQCHGELDPMVPVRFGALTAEKLRSVVTPARVQFKTYPGVMHSSCPQEMAAVKEFLEKLLPPV

SEQ ID NO: 6 - protein, 231 aa, Mus musculus
MCGNTMSVPLLTDAATVSGAERETAAVIFLHGLGDTGHSWADALSTIRLPHVKYICPHAPRIPVTLNMKMVMPSWFDLMGLSPDAPEDEAGIKKAAENIKALIEHEMKNGIPANRIVLGGFSQGGALSLYTALTCPHPLAGIVALSCWLPLHRNFPQAANGSAKDLAILQCHGELDPMVPVRFGALTAEKLRTVVTPARVQFKTYPGVMHSSCPQEMAAVKEFLEKLLPPV

SEQ ID NO: 7 - DNA, 3448 bp, Homo sapiens
acttccggcgctcgcaccgccccgctctccagccaaggctccgggctgaggcatttgcttggctgcagcctccttccgacctgcccggcgggacccaggggaccaagccgagccgagccgcggggcccgctccagcccggccatgagcgcggccgcatgatgcgtccctgcctcggccgctgcagtcgccgccgccgccgccgcaggccgggaggagccgcagcgccgggcgaccccgcccgggcctcggatccgatcacataggacagtatgcaccttaagatcctgaagaaacggcacaaaatgttcaagtgatgtttagaaataacttgtgagggtgcgtcagggaaatcatgcagccatcaggacacaggctccgggacgtcgagcatcatcctctcctggctgaaaatgacaactatgactcttcatcgtcctcctcctccgaggctgacgtggctgaccgggtctggttcatccgtgacggctgcggcatgatctgtgctgtcatgacgtggcttctggtcgcctatgcagacttcgtggtgactttcgtcatgctgctgccttccaaagacttctggtactctgtggtcaacggggtcatctttaactgcttggccgtgcttgccctgtcatcccacctgagaaccatgctcaccgaccctggggcagtacccaaaggaaacgctacgaaagaatacatggagagcttgcagctgaagcccggggaagtcatctacaagtgccccaagtgctgctgtattaaacccgagcgcgcccaccactgcagtatttgcaaaagatgtattcggaaaatggatcatcactgcccgtgggtgaacaattgtgtaggagaaaagaatcaaagattttttgtgctcttcactatgtatatagctctgtcttcagtccatgctctgatcctttgtggatttcagttcatctcctgtgtccgagggcagtggactgaatgcagtgatttttcacctccgataactgtaatcctgttgatcttcctgtgccttgagggtcttctgtttttcactttcactgcagttatgtttggcacccaaatccactccatatgcaacgacgagacggagatcgagcgattgaaaagtgagaagcccacatgggagcggaggctgcgatgggaagggatgaagtccgtctttggggggcccccctcactcctctggatgaatccctttgtgggcttccgatttaggcgactgcccacgagacccagaaaaggtggcccggagttctcagtgtgaggcgtggctcatcagactgaaacttgctcacagacttccagttatttatttggggtctgaaggatatcaacagctcatctgtgaccaacagggcaactggaacctacacaaaccaattgcttgcagcaagcagagttttatatatttatagtcacagatggcagaggaagaggctctcagtccccacctgtacaacaacggaaaggtgtgtggccacacgaagaagccaaacgccgtggcctcctgcagagctggggcttctgtggagaatacttcgggttattacatgggttattcaaatcctgggtcctgagctgctgtttccaatcatgaagaaaaacagtgaatccagtgaacagggattctccaagcagtcatttcagggggctcctgctgaccccgccactcagcagtgcactccccggatcacagcagggcgtttacatagaaagacgttttggtctcgattagctccgatgctttgcactgaagttgcaaaagatctgtgcactgaacagtgaaggtggcttccggcacactccccgctgccccggaagagacatcctttgaccctctcagcaagtctgtgtgtgtgcgtgtctgtgcgtgtgcgcgcgtgtgtgcatgtgtgtcaaaattgccagtgttgtttaggcaatgtaacatttaccggctgtgtacagcaaacaagctattttttagaaaccgacgtttcagggaagaggggagagagccgcggggtcctgcccgtggttactatgaatgtattgctgttggaggacatctcgatccaaagaacagccgttcctgtgcggcccttcgttgccctcctgctttcattttttaaagaaatcttgagtgcttgagggccttggaactgattttttttttttgttccagccaaattagcagtgtataaatggcacctaggtaagagcagagctgcggctcggtgacttgatacttggggcagcccgatgctgtgtgtggggcaggggaggcatccttactggagaggcagggcccagccattgggcacctctgggaaggggaggggaccatgaggcagccagcccctggcaggggcgactgtgccaccgcaggcagcgctccagtcgggaatggccaggatggcgccctcttgttggagtttttggttagcttttacgttttcttctccacccacggcacaggtgataaaataggatccttggtgcggagcttaaaattatgccagaaagccaacagctcccctcgtggggccttgccttaaacttgcctggtttgtacattttttgccggacgcatcaagaagcaatctgtgacaaagtctgagggtcttcctttatgcttgccctccacactaagagaagttggcgtctccctcctgggaattgttttgcctttctgttcatctgtgaactgttttttgtttttaattactctgtaccccatccgaatcagggcttctaccactgctgatgcaaaaccacaaagggacctacctgagccaccgtcctagccaagcgagcaaacctgcagggggtttggaagtggacttggtcaccgcagaagcgtgtgcgccgttgggggaagagctgcgtcacagccagagggacaaagtgtgggtgatcctggagacgccagtttccgagattgttctgcatattcatttgcacattgttgtctgggttggacatgcgtgtgggcttcagtgtgaggcttttaatatgtatatcctgttatcaataaaacaattatccaagtggttgaatcctgtgagacttggcaagtgtgtgcaaatcaagtatacttgacttttcaacctcttctttcaatgtaacttttatatgaaataaagtaatcaattaacagttctcaaatggcttccaaagcgcccgttcttcctggatggtggtgttttcagggggtgcctttccgtgcttcgtgggcccagccccaggctgttaaggtcactcccaaacagtttccagaaactctaaatgcttttgtgcccatgacaagtgacctgggcactcaagcgcgactcacaaatgttgcccagtgagtactgttgcttgttgaatgtttaattcgactcccatcgttttacttagtcctttttttagtgcacgttgaaataaaaacaatgtttcaaaatctccaca

SEQ ID NO: 8 - DNA, 2671 bp, Mus musculus
cagcctgggacagagcaagtcccaagtaaagaacaggttaggtccaggcctggtggtacacacctttaatctgggccacaccttctgctggagccctgcataagaaccgtggaggaagcaaggcttctcctccgcctgcttgcacttacttgccagcacatctgttggaagctacttcttcaggaacccggctcatacagaagagcattgagacacccagcctcacgggactgagctgctaaccagattcttggagttcccgtttgcatctgcccgttgttagggtgtttcagggaatcatgcagccgtcgggacacaggctccgggacatcgagcaccatcctctcctgactgacaatgacaattacgactcagcatcctcttcgtcctccgagactgacatggcagacagggtgtggttcatccgagatggctgtggcatggtctgtgctgtcatgacgtggcttctcgtcgtctatgcagacttcgtggtgacctttgtcatgctgctgccttccaaagacttctggtactccgtggtgaacggagtcctcttcaactgcttggcggtgctcgcgctgtcctcccacctgagaaccatgctcactgacccgggtgctgtccccaaaggcaacgccacgaaggagtacatggagagcttgcagctgaagccaggcgaggtgatctacaagtgccccaagtgttgctgcatcaagccggaacgtgcccaccactgcagtatttgcaagagatgcattcgaaagatggaccatcactgcccgtgggtgaacaactgcgtcggggagaagaatcagaggttcttcgtgcttttcaccatgtacatagctctgtcttcggttcatgctctgattctctgtgggcttcagttcatctcctgcgtccgagggcagtggacagagtgcagcgacttctctcctcccataactgtaatcctgttggtcttcctgtgccttgagggcctcctgttcttcaccttcaccgcagtcatgttcggcacccagatccactcgatatgcaatgatgaaacagagatcgagaggctgaagagcgagaagcccacgtgggagcgcaggctgcggtgggaaggaatgaagtctgtcttcgggggccctccctccctcctctggatgaaccccttcgttggcttccgactcaggcggctgcagatgaggaccaggaaaggaggccccgagttctctgtctgagccttcagggtgggaatgtctgcagacatccgtgctcagtggcctgcgtgcacagcagccttgtgggcagatggagcgggccagctgacggggccatgagcggcttcatggaccacaggccacaggcacttgtcatttctcttgtgtactgccaggagaagcatgcagtcccgggaggccatcgtctcaccatcctgacccttgggatgtcgcgtgggcacccacgggtggagttgcttctcagtcatgaagaaagccagcacagcagcgctgggagtcctgctggcccatccccagcgcacccccttacccgaaagaaggggttccacagagactctgccttggaggcctctgacacccagcactgaacagtgaaagcagcttccagaacactccctgctgccccggaacgggcatcctttgacccttccagcaagtctgtgtgtgctcatccttgtgtgtctgtgtggtgtgtgtgtgtgtgtgtgtgtgagcatgtgtcagagtcagcagtgtcgttgaggcacgtatgtggtagacaagctgtctttcagactccagtgtttcagggaagtgggacgcactgctggcccttgctggcctactatgaatgtatcctacagtgtccagatccaaagaacaggcatgcctgtgcatgggcccggcctcctgcttttatttcactgaaaatttagtgtttgagggcgtggagctgaggttttgttgttccagccaaactgcaactgcaacagtctctcaacagggagagtggtagcccatgctgaggcccagcgctttggggcaggggccatgaggagctcagcgtgagaagccacctagggccccttggggcaggtgctgcagggtgagacagcctggtgcctgagctgtcagagtggtgatatgtgactatttctctacccatggcaccgcagtcagtggagccttggtgcgggcttagaatcccgtcagtacacaaacagctcccctcaggggatttgccttaaccgctggttgttgctttgctgctgggtgagagcagtctgacagagtctggggacttcctgagagtcttctcccatgcagacaccagtgtttcccttctgggaccagctgcctttctgtccctgggacaccatgactgttctttcggtgtcagttgcttgtgccctgccctcatctgggctcctgtgctactgaggtgacccccagggacctgagccgagccctcatgcagcgagcaatctctggggcccaagtccctgcagagggacatcgaggcctcagattgtctctgcgttcctttgcacacgtgtctgggcatgtaggctctggcggtctcaccctccagtgtggagtctgtgcggtgaacccaagctgagtatacttgacttttcaattcctcagtgtgattttatatgaaataaagtaacccgtccacagtt

SEQ ID NO: 9 - DNA, 12594 bp, Homo sapiens
aacctgcgcggcggccgctcctgcagccgcggccgccgccactgccgggagagctcgatgggcttctcctgcgcgccgcccggtgtctggccgagtccagagagccgcggcgcctcgttccgaggagccatcgccgaagcccgaggccgggtcccgggttggggactgcaggggaaggcagcggcggcggcggcgggagccccaccggggtctgggactggggaactgcctccggcttcacgatgccagtatggacagaatagcttatgatgcttatccccacccaccacttccgaaacattgagcggaaaccagaatacctccagccagagaagtgtgtcccacccccctaccctggtcctgtgggaaccatgtggtttatccgtgacggctgtggcatcgcctgtgccatcgttacctggtttctggtcctctatgcggagttcgtggtcctctttgtcatgctgattccatctcgagactacgtgtatagcatcatcaacggaattgtgttcaacctgctggccttcttggccctggcctcccactgccgggccatgctgacggaccccggggcagtgcccaaaggaaatgccactaaagaattcatcgagagtttacagttgaagcctgggcaggtggtgtacaagtgccccaaatgctgcagcatcaagcccgaccgagcccaccactgcagtgtttgtaagcggtgcattcggaagatggaccaccactgtccctgggtcaacaactgtgtaggcgagaacaaccagaagtacttcgtcctgtttacaatgtacatagctctcatttccttgcacgccctcatcatggtgggattccacttcctgcattgctttgaagaagattggacaaagtgcagctccttctctccacccaccacagtgattctccttatcctgctgtgctttgagggcctgctcttcctcattttcacatcagtgatgtttgggacccaggtgcactccatctgcacagatgagacgggaatagaacaattgaaaaaggaagagagaagatgggctaaaaaaacaaaatggatgaacatgaaagccgtttttggccaccccttctctctaggctgggccagcccctttgccacgccagaccaagggaaggcagacccgtaccagtatgtggtctgaaggaccccgaccggcatggccactcagacacaagtccacaccacagcactaccgtcccatccgttctcatgaatgtttaaatcgaaaaagcaaaacaactactcttaaaactttttttatgtctcaagtaaaatggctgagcattgcagagaaaaaaaaaagtccccacattttattttttaaaaaccatcctttcgatttcttttggtgaccgaagctgctctcttttccttttaaaatcacttctctggcctctggtttctctctgctgtctgtctggcatgactaatgtagagggcgctgtctcgcgctgtgcccattctactaactgagtgagacatgacgctgtgcgtggatggaatagtctggacacctggtgggggatgcatgggaaagccaggagggccctgacctcccactgcccaggaggcagtggcgggctccccgatgggacataaaacctcaccgaagatggatgcttaccccttgaggcctgagaagggcaggatcagaagggaccttggcacagcgacctcatcccccaagtggacacggtttgcctgctaactcgcaaagcaattgcctgccttgtactttatgggcttggggtgtgtagaatgattttgcgggggagtggggagaaagatgaaagaggtcttatttgtattctgaatcagcaattatattccctgtgattatttggaagagtgtgtaggaaagacgtttttccagttcaaaatgccttatacaatcaagaggaaaaaaaattacacaatttcaggcaagctacgttttcctttgtttcatctgcttcctctctcaccaccccatctccctctcttccccagcaagatgtcaattaagcagtgtgaattctgactgcaataggcaccagtgcccaacacatacagccccaccatcatccccttctcattttataaacctcaaagtggattcactttctgatagttaacccccataaatgtgcacgtacctgtgtcttatctatattttaacctgggagactgttgtcctggcatggagatgaccatgatgctggggttacctcacagtccccaccctttcaaagttgacatatggccatcccattggccagaatccacagacacacctaagcctgtggcactgggacagaatagattttccatttgagaggcacttcctgtgtcagtcttgtttgaaggaggtggtgatggtggatagaggtgaaggaggtagggagtgccctccaagtgcaaaaataacaaatatgattattgaccatcggggaattctcacacattgatttgttttttaagcaattgccagaaacccccttttttagcttttgcttggggtgggggtaggagttaaggtttattcaatcctgtcctgggtagggcgaaagttaatctagccatgtgatttttcagaaaagtaagtggaacatgctgccacttttcaattctgtcagtgcttccacatggaaacaaaatgcaataaaatttttccaaaacctgttctgatttagctctctcttgaggtgttacccttagtgggaggccgactatccacaatctacttgagttttctctggttgggtgtttgtttcattgctctgtctcttgaatgaggatactttattttttttgttttaaaatgcatttatggtccctctcttgaaccagcttgccccaccaggcctctttcctttgctttctgcagcctgaatcaattcctttgtgctgatgggctctcctaagagctttcctgagtcagttaactttacctcgtgtctacggtgctattcatgcgatacgggcgaggctgagatgctaagattaaaaagaaaagaatgctgttttagatcaagttgatagcatttgttttccatatgcttttttaaaattttttcataacatacagctcagttaggtgtatgaaagaagtgttattgtattaaataactagagcagggctacagctctggccctcccctagggggaagagattggtaatactccatcttccagggcattttttaaagtgagccaggttagctcttttcccctggcatttctcaggaatgcagtagatagtgctgaagatgcactgacttttttttagtcctaaaaatagaaactcctcctttaaagctgtgcatactatgcttatctttccaatagagtggggttccttcagatatcctataggattctgcctctggttttgtataggccttggctagaaagagtcaatgtttctgagctctcaaaccagttgctctcagaagataggaataccccaaggttcctggcatttttcctatttcatttttgttcagactgatattttgccaagagcacaatgactgaggaatgtagccatcatttgcagggtagtgattggttcccagcctggcttccacacaggacaggaagggaaagcatccctgagctctcctcagtatttccggatgtaatgaaagaggacatctttctacacaaagtcagccccaacttttggcttggtcacaggagttctgatagtactgtttggtgcactcatgggaaattgaaccagtcgtagccacagtctttcagagcctgggctctggggagtggaagtgaaaaataaagatgtggcttgttggattgtgatccccagcttgctttccttctgtcaactctgtcaggtttgtgttcatagcaactagactgaatatgcaaaaggcttagatccaagcaaatctataatctatgcatatttgcatgggcttggtaatatcatgtacacaaaacacatttgggtagaagtgcatgtgctaaatctccttttagtcccaccattttgtcttcttcatactgtacttcctcttttttgtttgagacaaggtcttgctctgtcacccaggctggaatgcagtggcacaattagagctcactgcagccttgaactcctgggctcaagtgattcttgtgccttggcctcctgaatatccagggctacaggcacgtactaccatgcctggctaatttttttgttttttaatagagtcagggtctcactgtgttgccctagctagtctcaaatgcccggcctccagcaattttcctgccttagcctcccaaagtcctgggattacaggcgtgagccactgggcccagccctgtacttcttgaaaaagccccaagtattagcttttgctcatctggctaggccacttaaatagttagaatccaccgtcccctaatgcagaaaccgtttaggtgaggtaaattaacaaacattttaagccgggcgcggacacttctcactgtggacatccctcacgcctgtaatcccagcactttgggaggccgaggcgggcagatcacgaggtcaagagatcgagatcatcctggctaacacggtgaaaccctgtctctactaaaaatataaaaaaattagctgggcgtggtggcaggcgtctgtagtcccagctacttgggaggctgaggcaggagaatggcgtgaacccgggaagtggagcttgcagtgagccaagatcgcaccactgcactccagcctgggcgacagagtgagactccgtctcaaaaaaaaaaaacaaacattttaaacatgtatgtgaggttggcattacacagaaactcctctccgggtgggctgggatgggctttctcagccaggctaatgggttttaaatttctctcttttcaagacttgcagtgcatcagcttaaagggtgagccagccagtagaggggaaggcgccccacctagaaggtgcccttagatatcaaagaaatgtgaaaagagaaagattttgctagaatcctcctcaaaggtgttcttgaggttgccagaccagcaacgtcaacatcagcatcacctgagaacttgttagaaatgcacattctcggtccccaccccaggctaccgaaccagaaaccgagcggggcccagcagcccgtgtcttaacagccctccaggtgattctgactatcaagtttgagaatccagttggggctagcaggagtcccccctcaggtggtccctgatgcctgctggtgatatgggtcttgtgtgctgctgggctcagcatagtgcagttggggtgtgctgattgtgagacaggcacgtgttccctccgcggagaagccactgagactgccttccctcataagctgcggcctccccaacaaacaactgccaagacatcaaagaaagtctgtatgaagcagatccaaattattagcctgcccaccactccttgtgcatctcatcagtggaacccatctctagaccaagggccctttgggtgaagaagcagcccggaagggaaagagaaaagagtagaaccaagggacctccagatgggagcggcggccggtgagtagtctagagccaggggcattgtagcagcctggatacatgacctgaacacgtcttgacctttgctttctacgtgtgggtttcaacacccatgtggctttttcttgtattctttaaatatgtatctggcttaggatcacctcatagaagagaaagaattcacagtgaagcagaaacaagccactgaccagcgtactcccaacctgaaccttctttttctcaccctctccctcaagtaaacatcttgctgacttgagcagtgtgattgccgtagcaaagcagagtggcccccagggatcccgctctgttgggcccacaggaggagccgatgaagctgatccaaggagtgaggacaagcgctgcagagggacgttcgctaaaagccttctaggggccgcacatgctctaacacggacataaggatgccctgaatttctgcagctgaggccatatagtctggtgaccaagtatttgggtcctggcttcagtctttggttgaaatgtctgcttggctacttattaccgcacctactaccaaaatatgaccttgagcagtaacttctttaagcctcagttttttcatctgaaaacgggaatgataatctaaatcacaaagttaatggaaggattaaatgagggtgatgaataggaatgtatagcgtctggccctggtatggctttataaatgttagctgtgttggagctgtgcttttcaaaccattggtcacagccattcatggtttgcaaccagcatgtttttcaagaaaaatgtttaatgcattacatattgcaggataagtattgttttatgaagcttagggagttgtgtgtatatgtgttctggaatgcaacagaaaaatgtttcctcttgtgggttacaatatagaggtatgaaatctctgatgaggagagacagtgttatctggcccgctatgaagagacacatttgcataggctgctccctgaggctctggctttctacatctgatgatacagggagcagggaacagcctgttctcgttctgtggggctcagctgagtctgttctgcacagactcttccttcctcgggagccttagtcctaatacattcattttggagtgttggtgagtttgttcacagatcacagctcatgtgtcacccagactgacctgggccaaaaggcccatcacacaccctgcaagagcttctggtgtcgactatgacccccttaccaggcatcaaccatttttgttcgttctcttgagcctgaagctactattactgctcctctgcaaacctcaagcttaagaactttgcctgcaggatccctttaaatccacacaaaactcaaaattgagtcctaccaggaaaaagcagccctcagcccatttttatacatcggatttgtttgcaatattttctttctagactcaaaagtcaacactccctgaaagtttgtcgactttactgctgaagacctctggtagacaggccaggctctgtctggaatactttatgaggttggtgaggaggttgagtataatccaagagtgcctatctgggagcatgccacatgaatggcaaataatcatcctgtgggctcttggcttcattccccttctctctgactgagctcagcctgggcacagtggtgatttgcagtagaactggaaacctgttgggcagaaaaaaagacactagttctggttccagttctgatacataacaagctagatgagccttggccaccgtcatggcctcttggaacttctgtttcttccccatctgccaatcatcaatactcatacccacctcctcacaaggaggccataaaaacctatggtcatggctttgagtccaagtcagtgtggatgcagccagtctgtcatttttgggtgtttcctctgtagccgggtctgccatatggtgatgtcccagctctcgtgctatgaagttaaagcctctttctcaacaggctgcagatgatcacccaggaagagaatgcagaatgcccaaagcaaaccatctcagctggtcactgcttctgtgccaagaagggaggcctggcgaggggccagtcaggaagcagcatggcatcacatgctcatgacccacatgaaggtccctttagacttgtgtcaacaagatccattttctgaaacaactatttttgttctgattataaaagtaacattggctcattggtaaaacttggattgtgtgagaagtctacagaaataaatacaaatcctctagaattccatccccaaaagtaaccactcagacaaatgttctaatgtcatgtaaaaccatattaaaccatcttttctagctgcatagtgttatagaatcatttgcttaaccatcattattgggcatttctcatttccagctttgcattattataattcagtgttcaagtttgtattgcataaatctttgtctcagattattgattatttttaaactttttgtgaaatcagacttacaaaaatgtgacaaaaacagtacaaagagttcccatgtacctttcagtcagtctcaccaaaggtaaacattttatacaaccataatacaaatataaaaccctggacattggcaacaccatacccttaactaatgtatgtaccttattcacatttctccagttgtcccattaacacccttttctgttccaggatcccacactgcatcatttgcgatgtctccttagtctcctccagtttgtgacagttcctcagtcttcctttgtctttcatgaccttgaccctttttaaaaatcgaggtgaaattcctgtaacacaaaattagccattttaaagtgtacatttaatgcattcacaatgttttgtaaccaccaggtctgtctggttccaaaatcttttcatcaatctttgacccttttgaagattgtagggcaggtattctgtaggctgtccttcagattgtgtttttgatgtttttctcatgattagattgaggttaggcatttggggcaggagcactgctgaagcaatgtgtcctcgttgcaccgtatcaggaggcatatggtgttgatacgtttcattattgtgatgttaactttgatcattgggtgaaggtggtacgtgcaatgtttcttccctgctattaaggtactgtttttccctttgtaattgataagtatcttatgaggatatacttttgagatccaatttttttaacttagaatttattcaaaagtcaagaatcttaaatctctgaaatggcgtgggaagaaaaagtgctagatacacagagatctttcttgagtcatgtgaaggagcagtgcccaagcccagcaaacccacagcaaattcccttggcttccagaagagatggagaaagcagtgcccccagtggagggtcaaaggcctctgtgcagggtgttgtgggcctggagagctggcctggccatgtctttacctcctctgggcatctccccaccccaacaccctttctgtggcctggtggctgagttgcagccgacacccagaggcaggtgagttgacagcttggaagaggctgcagggtggatctgctgcatgagcaggcctgagcccagccttacctccccacagtggtcctgtgtgccctccggctgcctaatgcatgttggcacttgctgtacgagcacccgcttcttcacctcgcatgctgtttgtgtcctgcactccttccttaaccccatcgtccttctgctgtgtttgcagcccctatctaccctggtgggagtggccaaaaatatttaggaggggatcaccagtttgtagtggcctcagaggatgtgtggtcccccttatgcctcagccactcatcagcctagcccctgcccatcatctggcattgcacttgtggaaggaaagaaggggagggctgggtggtgggtggagaacacgtcagtccaccaggcgggccctgcttgctgtgttcctccacgctgctgtccacccacaccccagcagtcctctgagggacctcccgggggtgacctgggccacaacagactgcccactcagaccccatcttacccatgccgtggacaccccgcccccccccccgccactgctatgctatagctgggggtgtctatgtgagctgtacagcccagcaccacgctgacgatgttcttcatccccttctccctgcagggcatcgagcgcctcaaacgaaagaaccagcccagggagcacatggggagctggcagtcagtaaaggagacctttggtggggacttctccctgaactggttcaaccccttctccagaccgtgtcagccagagatccccagtgacaaagacatggtgcggcaggtgacatcgctgtcagacaccgaaacaatggaggatccatcagaggagacaaaggacgaggactctgtggaggtgacagatgaatagatgctgctgtggggagagaagcaaacactaaaaagtgctgtcaaccttcatcctggggttttggctaaaggggcttatgggcatggtgcgctcccagcacccccagtgcttcccttagccactcgcttggccttgccatttcccctccttcttctctccatgttgggccaggtctgggggtcgggagtaggctggggacatcagaggaggatgggggctttctcagagttcatctaagaagagtctgcactgagacggctcatcaagaaccgttctccaagactgggtggctttcacattctccgcccagcaaagggagcttttgaacagggcatcccaggggcagaaaagagcttgcctttggctttccccaggatttctgtcttctcttgggaaggctgggcccctggctcctggctttgagaagtaaggttgtgacagaaggaccgggcagggcttgccttggggacctgggttgggacactgacatcaggggagactagcctggaaagactgcagagctgccagctactccctggaaagggcttccccatgctgcctgccgaaattaggaggtagaggtggctgccacatctacctgcaagggccaggcatggttcaaagaggaccctgcattaagctctacacacacatgtgcaggacatgtccagcatggacagagccagagttaagacagtagcaccgaaaatgagcccccattccacagacactggagtcttcactgagcgagacagctgggagctgtcctgcctgtggctacatatctagccattcacagatgtggatatgggaaggacctctttggagctactggggactccctaaccactcgcatgagaacttaattgaatgttacctcttggagggagtctaataacacatgtaggtagaactgaccataaaccctgcctgtgtgtttgaaaaggccagttctcccaaattggtgcccatcttgtctctgaaaagatgggtgatggccagggtctgctgattgatgaatcagatgaatcaggaagatagacaaacacacacacacacacacacaccccaccaggatgagtctgccctctattcaccccatttgaagcctgtggtgtctgtgaccactgctgaaggtctgagcagcgttctggtgctcctaaaccccattccagtggttgctgaagcagcatcttctgcacaaagcccaacagaagggttcttatccccgtttggtataagaagtggattcaccacccactccctccacgtgcctttgttcctctctttggcccatttccccagcgtctactggcgtcaggattggcaggagcacaggcactcagcagagcatgcccctgcaagacctcagtgttagggccccccttccagctccaggcaaaagggcatgagtcctggccccaaggggcctgtggctgcagttcagaggagaagaaggtcagtgtttggaggtgcagcctcaggatgctgagaaaggaaactggcgaccgtgagaaagaaaagagccaagcagcatcctggttcttggacagcatctttggacactctgtgaagggcaacgatcctgccagagaccgtctctctacaactgatgacccactagggcctggggttaattgctcaaagggcccagtgttcacaaagccacctctgccctaacccttgccagagctctccaactatgacccacgagaggggtgatggtgggattctaacatcaacagagcaaccagaaagacattgggcctcccacactcaggctgcaggcccactttcttggtccttatcagctttaatatttattaatgacgacataggagcccgagtcagctgtaaaggccattaacttgcaatctggacaggaagttgacgctcaccactttgggtaagagctgctctgactgtagggccccctatttgttgtcctaacccagaagcagctctgggctgccaggatggtggatggaataccagagagttcacactagggaggaagcaatgcctgccccctggagtctcctagggggcagcagttagaataagggaagaggatttgctggtcactgtttgctgacatgggtttccatggtgagttcaggcctgaggacagcagtgtctgcaaaaccacatggcccttgagaaatgtccttgcacattgggcttcaaactcctcttctagggaatccatcttggcctgaaagcagaggtacaacaccagccccaaaggcaattctgttttcagattggttgctctggaaaggaaggctggggtgagggggcattttacttgcacagaggctgaccctgcctcccctcttcactgaccccatctccaaggtagacctcagccatgtcagtccctgttctgggaggtgctgggctgggccacagccagggttatgtaggtaattaacctgtccaaccctgagcctcgcctccccacaccagcaacacagtggtctctctgtggtgaccattcacagcataacattctgcttagcctcagactgaaagcattgcaactgatgtcaaaaccagatgagatcttacagggagagagattgggtgcaatttgcctctttctttgaataaaaagctctttgctcaccctca

SEQ ID NO: 10 - DNA, 5424 bp, Mus musculus
agcccgcgcggtggccgctcccgcagccgcggctgccgccaccgcctggagctccatgggcttctccttcgcaccgccccgtgtgaggccgagtccggcaagccgtggcgccgggtgcccgtgccatcgcggacgctggcggctgggccccggggcagcagcaggcggcgcgggactgctagctgcctccagattgacgatgccagtatggacagaatagcttatgatgcttatccccacccatcacttccgagacattgagcggaaaccagagtacctccagccagagaagtgtgccccacctcccttccctggtcctgcgggagccatgtggtttatccgagatggctgtggcattgcttgtgccattgtcacctggtttctggtcctctatgcggagtttgtagtcctctttgtcatgctggttccatcccgagactacgcgtacagcatcatcaacggaattgtgttcaacctgctggccttcttggccctggcctcccactgccgggccatgctgacggaccccggggcagtgcccaaaggaaatgccactaaagagttcatcgagagccttcagctgaagcctgggcaggtggtgtacaagtgtcccaagtgctgcagcatcaagcccgaccgggcacaccactgcagtgtttgtaagcggtgcattcgcaagatggatcaccactgtccttgggtcaacaactgtgtcggcgagaacaaccagaagtactttgtcctattcacaatgtacatagctctcatttccttgcacgccctcatcatggtgggattccacttcctgcattgctttgaagaagactggacaaagtgcagctccttctcgccgcccaccacagtcatcctgctcatcctgctgtgctttgaggccctgctcttcctcattttcacatcagtgatgtttgggacccaagtacactccatctgcacagatgagacgggtattgaacgcctccaacgaacaaaacagcccagggagcagtcgggcagctggaagtcggtccaggaggcttttggtgggaacttttccctgaactggttcaaccctttcaccagaccttgtcaaccagagacacccattgataagggcttggtacgtcaagtgtcatctctgtcagacgtggacaacatggaaacagctgagagccaattggaggcgacaaaagacagggactctgtggaggtgctggatgaatagaagtcatggaggataagacaccaacagtggctgctaccctgatgagtgttaccaactctgtcaccccacacttttttttccattaggattggggtcgggggataggaagggcaatagagggtacaggagagtagggactttctctgagttcctcttaacaggatgtgccaaaagtgatccagcaaaggaaatggtgaccaggatgtcggggtgggggatccacttctgcttcttgagggctgtcttggggaggctggggatcttggggaaggaatgctctaacatcaaggaagaccagcctggaaggctaaagagactccagccatttccaggaaagggattccttggctggctgactcgaaatcaacaaaggagggtatgctgagcctgcggtcagggcttgggcttggttgaaagagcaccctacatgcatgcatgcatacacacgtacagagtctacccaaatggaaatgagcctgagtcatgacagtatcgccatcctatagatacgagactcttccctgagacaacaaggcagtcagcctttctatagccacaagcatgcatcctagaagagctaggtgaagacatatattgaactcctggaaaaagtctgtaagacatggggacaggtgcaaccagaaatggcctcctttgcattttagagcagcgcccaaaatggtgcctcacagaaaactgcattgctagtcctaatgacatgccttattcccaacatttagagctggcaactaaacctctgttgctttgaggagaacctggtccacatggttctggctacatagggaaaccctgtcttaaaaacaatgaagaactgcatggtggccaggacatgcagactgttaagccaggaagaccccaggcactgctctcccaggatgagcttggacctagtctgtccactggagcttgaggtatctggctgttactgatagtctgggcaggctcctgcactccccagtccattgcagtggtggctgaagcggcagcttctgcagtctaaccagttagatagtctcaagaaacacacccagctcaactcaccccttgaaccattccccagcgtggctgccactgaccctggcagaggattgtatcacagctcctctagctctgcaaaagggcaggagtcctgggccagtggaacctgtgacctccatgacacgagtcaatcgaggtactgtctcaggaccctgagaaaggaaagctgtggggagccaagtggtggtctgattcttgggcagcatcctaggtagtgccacagtgtcagcaggacttcagcattagcagatgtggcctactccaccccactgcaagtgcacttcctccaagcaggtctgctttaacatttatcagcaataacatgaggaacccaagttgtccctaatcttggcactatgtggaaagacctgtagggcctccaaccccaaacagacctggacggcccaggtgctggaccataaccagagattccataccagggagaaatcagagctagtgactaggtccccaaggagcagcagttaaattaaggaaggagctttggagtcaggatttgttaactgaatgtcctgtacattgaggctaaaaccagtcttgcttggggacagcttgacctgcagcagagatacctcccagcccacagccatttgtttccagagtgcttttcctggaaaggttgaagtgtgcattttgctcccagttttaggtcctccatactcctcagctaacccattaccatgtcaggcctcagccatatttcttctgtgtactggacagccaggatcatgtaggtagtagtctgtccagccctgggccttgtgccttcacaccagaacccaatggccttctctctatggcaactcctgagattctactcagcactgcagctgataagaggaggcccagtgtttctgggccagaggctgcttgcacacctgcctcttgctttgaataatagttctttggtctacctctgtggctagaggcgccaattttttagcaggagatcctagcccaaagccttggacaacgtccttcctctcaggtctagtgtgtaaaacaagtgtcacaaacactaactgatcctccatggccccagcatagacagaggttattctacaagcatgtaccagacatctgcacgccacagaggtgggcatctgggcaaaaaggagtcaggacttggggttttctggtttcaagaatgaaggaaagatgacaggttttaaattaacttgacacaagctgaaggtatttgagaagagggaatctcaattgagaaaatgcctctgtaagattggcctttacacaaatgtgtagtgcattttcttgattgatgtaggagggccaggcccattgtcagttatgccacccctggatggaagaaaaaaacatgacgggcaagttatgaggagcaagccagtaagcagcactcctctgtgacctctgcattagcccatgcctctagggtcctgccctaacttccctcagatgcaattaaccctttcctccccaggttgctttaggtcgtagtgttttatcacagcaacagaaaccctaaggcaagaatggcctttaaatacacaaaataccccttattcagtgtgtactgagtatctctggaacacatgtccacttagaattacagcccttgagagaggttgaccatcacgaagaggacccagaccaagaaggccaacctggatccatgaaattcagaggcaatgaggttaaattaagcagcatgctggcataaaagaaaatttaactccagaccacttagccatcttaaagttgccagagtttaattcaaagcatagtggtatgtgagcagataccttaacagcagagcaaagcacagactcccgagaacagggcaggatacgttgcttcttcagatgttgtcactggccatagactcttacttaaaaaaagggtctacctcccatggctcatccagagcaagagggcctcacgccagcgtggggcctgcttctaagaggacagagccttgggataagagggtgtagtgcctgacccaggaccagctccaagatgctttctataagacttccagcacagcctccaggcacccctggacttgatagaatccatggtctagatggaagatgcctccagcccttctatatctgggctctgtacatggttctgaatccagatgacggtggcactgtcttctgaggccacaattcctaaatatgtatttgaaatgtttgcctgtgacatgtgcccaggttctctggctccctggagctactgcctgtggtgacatcggcctttgagctctgtcttgctgggctggctcttgaatgtgccttcacacgcataatcaaatttaaccatccctgacctgaaagctaaatgcaatttagaaaatgttaacatgcttagccaaggctcagagccaatcagggctcctgcctccatctccatggtatcaaatgtgctacttgctgatgagaacgaccttctcggcatgaacctctttcacttgtctggaaataaacttgacaatgaatttctggggtccagctttcactggggtgcattccatttggaaagtgatggtctttccaggtggcactgtcctagaaaattcaaaggcacacagtcagccaaaagtgccacagagagaaaatcgcattgcatgaggaaccaggcaggcatccggttccagtacagatcttgggaaatccctgtgctgcagagccactgaagcaggcctccacctggagggtggccacttgtctcagctgtaagacaagatggcctgctggaatggctgttttccagctatgccagtcacaaaccctcctgagagccctcaggaacaaattttatgatgaatctacaagaaagtgttgtctaatcctagtattcaagatggcaatacatgagagtatgttaggctctgaggccctgccccaatactactgaactgaatgtagttttgttgttgttgtttgtttattttggggtttgtttggtttggtttgagtttttttttttttttttggaggggggggatgtttcaagacagggtctttctgggtagccccagctgtcctagaactcactctgtagaccaggctggccttgaactcacaacggttcacctgcctctgcctccaaagtgctgagattaaaagtgtgcaccacca

SEQ ID NO: 11 - DNA, 1635 bp, Homo sapiens
gggactcgaacggaagttccggcgggggcggccgagggggaagagtgtgtctgcgggagaaagaggagaatcgcccaagcggcctcggaagtcccagggagtggaggcccccgccgtggagccgtgtggtgtatgtgtggtaacaccatgtctgtgcccctgctcaccgatgctgccaccgtgtctggagctgagcgggaaacggccgcggttatttttttacatggacttggagacacagggcacagctgggctgacgccctctccaccatccggctccctcacgtcaagtacatctgtccccatgcgcctaggatccctgtgaccctcaacatgaagatggtgatgccctcctggtttgacctgatggggctgagtccagatgccccagaggacgaggctggcatcaagaaggcagcagagaacatcaaggccttgattgagcatgaaatgaagaacgggatccctgccaatcgaatcgtcctgggaggcttttcacagggcggggccctgtccctctacacggccctcacctgcccccaccctctggctggcatcgtggcgttgagctgctggctgcctctgcaccgggccttcccccaggcagctaatggcagtgccaaggacctggccatactccagtgccatggggagctggaccccatggtgcccgtacggtttggggccctgacggctgagaagctccggtctgttgtcacacctgccagggtccagttcaagacatacccgggtgtcatgcacagctcctgtcctcaggagatggcagctgtgaaggaatttcttgagaagctgctgcctcctgtctaactagtcgctggccccagtgcagtaccccagctcatgggggactcagcaagcaagcgtggcaccatcttggatctgagccggtcgagcccctgtccccacccttcctgacctgtccttttcccacaggcctctgggggcaggtggcaaggcctggccgggccttccttcctggccttagccacctggctctgtctgcagcaggggcaggctgctttcttatccatttccctggaggcgggcccccctggcagcagtattggaggggctacaggcagctggagaaaggggcccagccgctgacccactcactcaggacctcactcactagccccgctttgggccccctcctgtgacctcagggtttggcccatggggcccccccaggcccctgccccaactgattctgcccagataatcgtgtctcctgcctccactcagctgcttctcagtcatgaatgtggccatggccccggggtccccttgctgctgtgggctccctgtccctgggcaggagtgctggtgaggaggtggagccttttgaggggggccttccctcagctgtttccccacactggggggctgggccctgcctccccgttaccctccttccctgcaggcctggagcctgtagggctggactgaggttcaggtctccccccagctgtctcacccccactttgtccccactctagagcagggaggcagtgggggaggagttgtgtctcgtcttctgtctccatgtggtttttgggtgtttttcttgttgtgtcctggattccgataaaattaaagaaattgcttcctcaa

SEQ ID NO: 12 - DNA, 1593 bp, Mus musculus
cgagggggaagagtgtgtgcctgtgcgggagaaaggggagaatcgcccgaacggtctcggaagccactgggagtgaggcccagcctgcaggaagccctgcagtgtatgtgtggtaacaccatgtctgtgcccctgctcaccgacgcagccaccgtgtctggagctgagcgggaaacggccgcggttatttttttacatggacttggagacacagggcacagctgggctgacgccctctccaccatccggcttcctcatgtcaagtacatctgtccccatgcgcccaggatccctgtgacactcaacatgaagatggtgatgccctcctggtttgacctgatggggctgagtccggatgccccagaggatgaagctggcatcaagaaggccgcagagaacatcaaggctttgattgaacatgagatgaagaacgggattcctgccaaccggatcgtcctgggtggcttctcgcagggtggggccctgtccctctatacagcacttacctgcccccaccctctggctggcattgtggcattgagctgctggctgcctctgcaccggaacttcccccaagcagccaatggcagtgccaaggacctggccatccttcagtgccacggggagctggaccccatggtacctgttcggtttggagccctgacagctgagaagctccggactgttgtcacacctgccagggtccagtttaagacatacccaggtgtcatgcacagctcctgtcctcaggagatggcagctgtaaaggaatttctggagaaacttctgcctccggtctaactagttgctggctcccaccatggtcccccagctcatgggggacccagcaagcaagcaccgcatcatcttggatctgagccggtcgagcccctgtcccttctcttcccaccctatcctcttcccacaggcctctggggcaggtcgccgaggctggccaggccttcttgctggccttggccatctggaagtcagcagcggggtgggctgctctcttatcctgcgtccccagaggcgggccccagcagcagtattggaggggctgtaggcggcgggaggaaggggcccagctgctgacccgctcactcaggacctcacccactagccccgctttgggccccctcccgtgacctcagggtttggcccgtggggccctcgtgggcccctccccccatgactctgcccggataatcgtctctcctgcctcgctgcagccgcttctgtcacgaatgtgtccatggcccggggcccttgctgctgtgggctgcctgctccaggacaggctggccagtgaggaggtagagtccctcggggcctcccctcagcccctccccgagaggggctggaccctgcctccccatggcccttccttgcaggcctggagcctgcagggctggattgaggttcaagggcccccccaactccctgctgtctcacccccactatgtccccccttagggccagggaggtggtgggggaggagttgtgtcttgtcttctgtcttcatgtggttttgggtgtttttctcgttttgtcctggattccgataaaattaaaggaattgcttcaactctc

SEQ ID NO: 13 - DNA, 23 bp, Artificial Sequence
  Oligonucleotide
tgagccaggatggatttcagaca

SEQ ID NO: 14 - DNA, 23 bp, Artificial Sequence
  Oligonucleotide
tgccctcggacgcaggagatgaa

SEQ ID NO: 15 - DNA, 23 bp, Artificial Sequence
  Oligonucleotide
tcccctgatgtatgcgaatgtcc

SEQ ID NO: 16 - DNA, 23 bp, Artificial Sequence
  Oligonucleotide
aacaggtgccttttgaatgtcag

SEQ ID NO: 17 - DNA, 25 bp, Artificial Sequence
  Oligonucleotide
caccggaggatgatgctcgacgtcc

SEQ ID NO: 18 - DNA, 25 bp, Artificial Sequence
  Oligonucleotide
aaacggacgtcgagcatcatcctcc

SEQ ID NO: 19 - DNA, 25 bp, Artificial Sequence
  Oligonucleotide
caccgcgtcgagcatcatcctctcc

SEQ ID NO: 20 - DNA, 25 bp, Artificial Sequence
  Oligonucleotide
aaacggagaggatgatgctcgacgc

SEQ ID NO: 21 - DNA, 25 bp, Artificial Sequence
  Oligonucleotide
caccgcgggtctggttcatccgtga

SEQ ID NO: 22 - DNA, 25 bp, Artificial Sequence
  Oligonucleotide
aaactcacggatgaaccagacccgc

(19) United States
(12) Patent Application Publication
(10) Pub. No.: US 2023/0159931 A1
(43) Pub. Date: May 25, 2023

(54) TARGETING THE PALMOTYLATION/DEPALMOTYLATION CYCLE TO TREAT INFLAMMATORY DISEASES

(71) Applicant: CORNELL UNIVERSITY, Ithaca, NY (US)

(72) Inventors: Hening LIN, Ithaca, NY (US); Mingming ZHANG, Ithaca, NY (US); Tao YU, Ithaca, NY (US)

(73) Assignee: CORNELL UNIVERSITY, Ithaca, NY (US)

(21) Appl. No.: 17/919,650

(22) PCT Filed: Apr. 23, 2021
(86) PCT No.: PCT/US2021/028811

§ 371 Date: Oct. 18, 2022

Related U.S. Application Data
(60) Provisional application No. 63/014,735, filed on Apr. 24, 2020.

(51) Int. Cl. C12N 15/113; A61P 29/00; C12N 9/22

(52) U.S. Cl. CPC C12N 15/1137; A61P 29/00; C12N 9/22; C12N 2310/14; C12N 2310/20

(57) ABSTRACT

This disclosure is directed to methods for treating an inflammatory disorder, comprising administering to a patient suffering from the disorder an effective amount of an inhibitor of an enzyme that regulates the S-palmitoylation of a pro-inflammatory transcription factor.

### CROSS REFERENCE TO RELATED APPLICATION

This application claims the benefit of priority from U.S. Provisional Application No. 63/014,735, filed Apr. 24, 2020, the entire contents of which are incorporated herein by reference.

### STATEMENT REGARDING FEDERALLY SPONSORED RESEARCH OR DEVELOPMENT

This invention was made with government support under Grant Nos. R35GM131808 and R01GM121540, awarded by the National Institutes of Health. The government has certain rights in the invention.

### INCORPORATION BY REFERENCE OF SEQUENCE LISTING

The Sequence Listing in an ASCII text file, named as 38342WO_9346_02_PC_SequenceListing.txt of 52 KB, created on Apr. 20, 2021, and submitted to the United States Patent and Trademark Office via EFS-Web, is incorporated herein by reference.

### BACKGROUND

Cysteine palmitoylation (S-palmitoylation) is an important protein post-translational modification (PTM), regulating protein membrane associations and protein-protein interactions. S-Palmitoylation is catalyzed by 23 palmitoyltransferases, known as DHHCs due to the conserved Asp-His-His-Cys sequence motif, and is removed by acyl-protein thioesterases (APT1, APT2, and ABHD family members). Although thousands of human proteins are known to undergo S-palmitoylation, how this modification is regulated to modulate specific biological functions is poorly understood.

Treatment options are limited for chronic inflammatory diseases, such as inflammatory bowel disease (IBD), which includes ulcerative colitis and Crohn's disease. Although the aetiology of IBD is unknown, the association between IBD and immune dysregulation has been extensively studied. The abundance of proinflammatory cells in patients with IBD contributes to its progression, and blocking the differentiation of proinflammatory cells could be useful in treatment of the disease.

As a potent immunosuppressive cytokine, transforming growth factor beta (TGFβ) suppresses the maturation and functions of anti-inflammatory effector/memory immune cells by inducing the differentiation of regulatory T (T_{reg}) cells from naive CD4⁺ T cells. However, combined with Interleukin-6 (IL-6), TGFβ promotes the differentiation of proinflammatory T helper 17 (Th17) cells from naive CD4⁺ T cells. Th17 cells are characterized by the expression of retinoic acid receptor-related orphan receptor gamma t (ROR-γt, gene name RORC) and Interleukin-17 (IL-17, gene name IL17A). Many immune related diseases such as multiple sclerosis (MS), inflammatory bowel disease (IBD) and rheumatoid arthritis (RA), are chronic inflammatory processes with imbalanced T_{teg}/Th17 cells ratio, suggesting that T cells differentiation plays a key role in immune diseases. Due to the important role of the TGFβ signaling pathway in T cell differentiation, understanding how the opposing functions of TGFβ is regulated could be useful for treating immune related diseases.

T_{H}17 cells are a subgroup of proinflammatory T cells that are characterized by the expression of interleukin-17 (IL-17, encoded by IL17A) and retinoic acid receptor-related orphan receptor gamma t (RORγt, encoded by RORC). Accelerated differentiation of T_{H}17 cells has an important pathogenic role in IBD, and the abundance of T_{H}17 cells correlates with disease activity in mouse models of IBD and in patients. The serum of patients with IBD is rich in cytokines, enabling the differentiation of T_{H}17 from naive CD4⁺ T cells. Under specific cytokine stimulation, STAT3 in naive CD4⁺ T cells is phosphorylated by Janus kinase 2 (JAK2). As a key transcription factor, p-STAT3 promotes the expression of downstream target genes (RORC and IL17A) and the differentiation of T_{H}17 cells. Owing to the important role of STAT3 in T_{H}17 differentiation, understanding how STAT3 is regulated could provide new ways to control T_{H}17 cells.

The intracellular transduction of the TGFβ signal is initiated by the binding of TGFβ to TGFβ receptor II (TGFβRII) on the cellular membrane. Subsequently, TGFβRII phosphorylates and activates TGFβ receptor I (TGFβRI). As primary responders to TGFβ, mothers against decapentaplegic homolog 2 and 3 (SMAD2 and SMAD3) are highly homologous TGFβ receptor-regulated SMADs (R-SMADs). They can be recruited and phosphorylated by TGFβR at two serine residues on the carboxy-terminal (C-terminal) (Ser465/Ser467 for SMAD2 and Ser423/Ser425 for SMAD3). SMAD4, as the only known human Co-SMAD, partners with R-SMADs to recruit transcriptional co-regulators to the complex. Although SMAD2 and SMAD3 share the same upstream signaling pathway and many downstream partners, they have opposite functions in T cell differentiation, with SMAD2 promoting Th17 and SMAD3 promoting T_{reg}. A previous study suggests that the phosphorylation of Ser255 of SMAD2 by extracellular signal-regulated kinase (ERK) promoted its interaction with STAT3, then the SMAD2-STAT3 complex translocates to the nucleus to promote Th17 cell differentiation, whereas the carboxy-terminal unphosphorylated SMAD3 interacts with STAT3 to repress the Th17 cells differentiation. This finding suggests that SMAD2 Ser255 phosphorylation could tune the Th17-T_{reg} balance, but how this phosphorylation process is regulated is largely unknown. Understanding the regulatory mechanisms may provide new therapeutic strategies to control the Th17-Tr_{eg} ratio and treat immune related diseases.

Mothers against decapentaplegic homolog 2 and 3 (SMAD2 and SMAD3) shared the same primary signal pathway in response to transforming growth factor beta (TGFβ), yet they have opposing effects on T cell differentiation, with SMAD2 promoting pro-inflammation T helper 17 (Th17) while SMAD3 promoting anti-inflammation regulatory T (T_{reg}) cells. How their opposing T cell differentiating functions are achieved and regulated remains to be understood and such understandings can help to treat inflammatory diseases.

### SUMMARY OF THE DISCLOSURE

An aspect of the disclosure is directed to a method for treating an inflammatory disorder, comprising administering to a patient suffering from the disorder an effective amount of an inhibitor of an enzyme that regulates the S-palmitoylation of a pro-inflammatory transcription factor.

In some embodiments, the enzyme is Zinc finger DHHC-type palmitoyltransferase 7 (ZDHHC7) or Zinc finger DHHC-type palmitoyltransferase 3 (ZDHHC3).

In some embodiments, the enzyme is lysophospholipase 2 (LYPLA2).

In some embodiments, the inhibitor of the enzyme is a nucleic acid inhibitor.

In some embodiments, the nucleic acid inhibitor is selected from the group consisting of an antisense RNA, a small interfering RNA, a microRNA, an artificial microRNA, and a ribozyme.

In some embodiments, the inhibitor of the enzyme is a genome editing system. In some embodiments, the genome editing system is selected from the group consisting of CRISPR/Cas system, Cre/Lox system, TALEN system, ZFNs system and homologous recombination.

In some embodiments, the CRISPR-mediated genome editing comprises introducing into the patient a first nucleic acid encoding a Cas9 nuclease, a second nucleic acid comprising a guide RNA (gRNA), wherein said gRNA is specific to the gene encoding the enzyme.

In some embodiments, the inhibitor of the enzyme is a small molecule inhibitor.

In some embodiments, the enzyme is LYPLA2, and the inhibitor is ML349 with the following chemical formula:

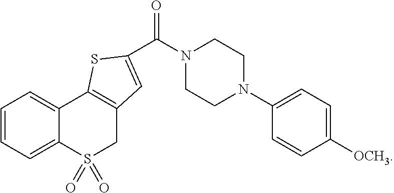 [SMILES: COc1ccc(N2CCN(C(=O)c3cc4c(s3)-c3ccccc3S(=O)(=O)C4)CC2)cc1]

In some embodiments, the enzyme is a Zinc finger DHHC-type palmitoyltransferase, and the inhibitor is selected from 2-bromopalmitic acid, cerulenin or tunicamycin.

In some embodiments, wherein the disorder is an autoimmune disorder. In some embodiments, the autoimmune disorder is selected from the group consisting of inflammatory bowel disease, multiple sclerosis, rheumatoid arthritis, lupus, graft versus host disease, type I diabetes, gout, asthma and psoriasis.

In some embodiments, the disorder is an endotoxic shock, e.g. an LPS-induced endotoxic shock.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D. DHHC7 (synonymous with ZDHHC7)-induced palmitoylation promotes STAT3 membrane translocation. (A) HEK293T cells were transfected with Flag-STAT3 and HA-DHHC7. Palmitoylation levels of STAT3 with or without hydroxylamine (NH₂OH) treatment were detected using Alk14 labelling. (B) Left, the subcellular localization of endogenous STAT3 and JAK2 was analyzed using confocal imaging in wild-type and DHHC7-knockout HEK293T cells. Scale bars, 50 μm. Right, quantification of the colocalization of STAT3 and JAK2 using Pearson's correlation coefficients. (C) Left, the subcellular localization of EGFP-STAT3 and EGFP-STAT3(C108S) in DHHC7 knockout HEK293T cells ectopically expressing DHHC7. Scale bars, 100 sm. Right, the percentage of DHHC7-positive cells in which STAT3 is translocated from the nucleus to the plasma membranes and endomembranes. (D) Wild-type and DHHC7-knockout HEK293T cells were transfected with Flag-STAT3 and labelled with Alk14. Subcellular fractionation was performed and STAT3 protein levels were adjusted to ensure that there were equal amounts of STAT3 in the wild-type and knockout cell fractions used for the gel. The palmitoylation levels of immunoprecipitated STAT3 in the membrane (mem.), cytoplasmic (cyto.) and nuclear (nuc.) fractions were visualized by in-gel fluorescence. Data are mean±s.e.m. **P<0.01.

FIGS. 2A-2H. APT2 is a depalmitoylase of STAT3 and palmitoylation/depalmitoylation promotes p-STAT3 nuclear localization. (A) C108 is the major palmitoylation site of STAT3. HEK293T cells were transfected with HA-DHHC7 and Flag-STAT3 (wild-type or mutants) and labelled with Alk14. STAT3 was pulled down and subjected to Alk14 labelling and western blot analyses. (B) Left, distribution of wild-type or C108S mutants of STAT3 and p-STAT3 in the subcellular fractions of DHHC7-knockout HEK293T cells into which DHHC7 or DHHS7 were reintroduced. Right, quantification of the relative p-STAT3 levels. (C) The subcellular localization of STAT3 and p-STAT3 was analysed using confocal imaging after different EGFP-STAT3 constructs and HA-tagged DHHC7 were transfected into DHHC7-knockout HEK293T cells. Scale bars, 50 sm. (D) In HEK293T cells, overexpression of wild-type APT2—but not the C2S or S122A mutants—decreased the palmitoylation level of Flag-STAT3 as determined by Alk14 labelling. (E) APT2 preferentially depalmitoylates wild-type STAT3 over the Y705F mutant. DHHC7-knockout HEK293T cells were transfected with the indicated plasmids and labelled with Alk14. The palmitoylation of STAT3 was determined by in-gel fluorescence (left) and quantified (right). (F) APT2 inhibition or knockdown increases the palmitoylation of Flag-STAT3. DHHC7-knockout HEK293T cells were reintroduced with HA-DHHC7 and Flag-STAT3, and treated with LYPLA2 siRNA or 20 μM of ML349 for 36 h before Alk14 labelling and in-gel fluorescence detection. (G) Left, distribution of STAT3 and p-STAT3 in different subcellular fractions of APT2-knockdown HEK293T cells. Right, quantification of the relative STAT3 and p-STAT3 levels in these fractions. (H) Left, distribution of STAT3 and p-STAT3 in subcellular fractions of DHHC7-overexpressing HEK293T cells, with or without treatment with 1 μM of the JAK2 inhibitor fedratinib. Right, quantification of the relative STAT3 levels in these fractions. Data are mean±s.e.m. *P<0.05; **P<0.01.

FIGS. 3A-3E. The STAT3 palmitoylation-depalmitoylation cycle promotes T_{H}17 cell differentiation. (A) DHHC7 promotes phosphorylation of wild-type STAT3, but not of the C108S mutant, in mouse splenocytes. Left, STAT3 and p-STAT3 blots; right, quantification of the relative p-STAT3 levels. (B) T_{H}17 cell differentiation was quantified in the splenocyte samples in A by flow cytometry. (C) APT2 inhibition decreases T_{H}17 cell differentiation in a dose-dependent manner. Mouse splenocytes were treated with a cytokine cocktail (3 ng ml⁻¹ TGF-β, 40 ng ml⁻¹ IL-6, 30 ng ml⁻¹ IL-23, 20 ng ml⁻¹ TNF and 10 ng ml⁻¹ IL-1β) and different concentrations of ML349 for 4 days, then collected and analysed by flow cytometry to detect CD4- and IL-17-positive cells. (D) DHHC7 knockout in splenocytes inhibits T_{H}17 cell differentiation. Wild-type and DHHC7-knockout mouse splenocytes were treated with a cytokine cocktail for 4 days to initiate differentiation, then the cells were collected and analyse by flow cytometry to detect CD4- and IL-17-positive cells. (E) Left, STAT3 and p-STAT3 blots of wild-type and DHHC7-knockout splenocytes. Right, quantification of the relative p-STAT3 levels. Data are mean±s.e.m. **P<0.01.

FIGS. 4A-4I. The STAT3 palmitoylation-depalmitoylation cycle aggravates colitis. (A), (B) Human PBMCs from 26 healthy participants (Ctrl), 24 patients with Crohn's disease (CD; 7 remission stage (rem.) and 17 active stage (act.)) and 10 patients with ulcerative colitis (UC; 1 remission stage and 9 active stage) were extracted. LYPLA2 and ZDHHC7 mRNA levels were analysed using qPCR. The relative p-STAT3 levels were quantified by western blots. c, d, The correlations between IL17A (C) or p-STAT3 (D) and the mRNA levels of the indicated genes in 34 patients with IBD. (E), (F), C57BL/6J mice were treated with 2.5% DSS in drinking water ad libitum and ML349 was intraperitoneally injected daily on the day that DSS treatment started. Body weight changes (E) and T_{H}17 cell levels in the spleen (F) were evaluated. (G), (H), Wild-type and DHHC7-knockout mice were treated with 2.5% DSS in drinking water ad libitum. Body weight changes (G) and T_{H}17 cell levels in the spleen (H) were evaluated. (I), Model of the regulation of STAT3 by the palmitoylation-depalmitoylation cycle. Palmitoylation of STAT3 by DHHC7 promotes the membrane recruitment and phosphorylation of STAT3. APT2 promotes the nuclear translocation of p-STAT3 by selectively depalmitoylating p-STAT3 over STAT3. The palmitoylation-depalmitoylation cycle drives the membrane localization and phosphorylation of STAT3, and the nuclear translocation of p-STAT3. The direction of the cycle is ensured by the preference of APT2 for p-STAT3 over STAT3. Data are expressed as the mean±s.e.m. *P<0.05; **P<0.01.

FIGS. 5A-5G. SMAD2 is palmitoylated by DHHC7. (A) HEK293T cells were transfected with Flag-SMAD2 and indicated HA-DHHC plasmid and the palmitoylation level was detected using Alk14 metabolic labeling and in-gel fluorescence. All DHHC genes used were from mouse and that the protein number corresponds to the gene number with the exception of DHHC10/Zdhhc11, DHHC11/Zdhhc23, DHHC13/Zdhhc24 and DHHC22/Zdhhc13. (B) Quantification of the relative palmitoylation levels in A. The palmitoylation level was normalized by SMAD2 protein level and the level of control (with Alk14 but without DHHC overexpression) was set to 1. (C), (D) HEK293T cells were transfected with Flag-SMAD3 and Flag-SMAD4, respectively, and indicated HA-DHHC plasmid. The palmitoylation level was detected using Alk14 labeling and in-gel fluorescence. (E) HEK293T cells were transfected with HA-DHHC1-23 plasmid, and palmitoylation level was detected by ABE assay with NH₂OH treatment. (F) HEK293T cells were transfected with Flag-SMAD2 and Flag-SMAD3, respectively, and indicated HA-DHHC7 plasmid. The palmitoylation level was detected using Alk14 labeling and immunoblotting. (G) The palmitoylation level of SMAD2 in DHHC7 WT and knockout HEK293T cells was detected using ABE. Quantification data are expressed as the mean±SEM. Asterisks (*) indicate significant differences (**P<0.01).

FIGS. 6A-6F. Cys41 and Cys81 palmitoylation promotes the membrane recruitment of SMAD2. (A), (B) HEK293T cells were transfected with HA-DHHC7 and different Flag-SMAD2 cysteine mutants and labeled with Alk14. The S-palmitoylation levels of immunoprecipitated SMAD2 were visualized by in-gel fluorescence. (C) SMAD2 C41/81S mutant could not be palmitoylated by DHHC7. DHHC7 WT and KO HEK293T cells were transfected with Flag-SMAD2 C41/81S mutant plasmid and treated with Alk14. The S-palmitoylation level of immunoprecipitated SMAD2 C41/81S was visualized by in-gel fluorescence. (D) Confocal imaging showing the subcellular localization of SMAD2-WT and C41/81S mutant in DHHC7 KO HEK293T cells ectopically expressing DHHC7. Scale bars, 50 μm. (E) SMAD2 C41/81S mutation decreases SMAD2 cytosolic localization and increases nuclear localization. DHHC7 KO HEK293T cells were transfected with HA-DHHC7 and the indicated Flag-SMAD2 constructs and subcellular fractionation was performed. Equivalent amounts of the nuclear and membrane fractions were then analyzed by western blots (left). The relative SMAD2 levels were quantified (right). (F) SMAD2 S-palmitoylation in different subcellular fractions of DHHC7 KO HEK293T cells ectopically expressing WT or inactive mutant DHHC7. The cells were transfected with Flag-SMAD2 and labeled with Alk14. Cell fractionation was performed and the protein level of SMAD2 was readjusted by CBB to make sure the equal loading of SMAD2 in WT and mutant cell fractions for the gel analysis. The S-palmitoylation levels of immunoprecipitated SMAD2 in the membrane (Mem), cytoplasmic (Cyto), and nuclear (Nuc) fractions were visualized by in-gel fluorescence. Quantification data are expressed as mean±SEM. Asterisks (*) indicate significant differences (**P<0.01).

FIGS. 7A-7G. APT2 is the depalmitylase of SMAD2 and C-terminal phosphorylation of SMAD2 is S-palmitoylation independent. (A) C-terminal phosphorylation (Ser465/Ser467) levels of Flag-SMAD2 (p-SMAD2(C2)) in HEK293T cells expressing HA-tagged mouse DHHC1-23. SMAD2 was pulled down with Flag beads and subjected to western blot analyses. (B) HEK293T cells were transfected with HA-tagged DHHC7 and Flag-SMAD2 WT and C41/81S mutant plasmid and treated with Alk14. The S-palmitoylation level of immunoprecipitated SMAD2 was visualized by in-gel fluorescence and the p-SMAD2(C2) level was detected by western blot analyses. (C) HEK293T cells were transfected with HA-tagged DHHC7 and Flag-SMAD2 plasmids as indicated and treated with TGFβ. SMAD2 was pulled down with Flag beads and subjected to western blot analyses. (D) HEK293T cells were transfected with HA-tagged DHHC7 and Flag-SMAD2 plasmids as indicated and treated with 10 μM APT inhibitors for 24 hours as indicated. The S-palmitoylation level of immunoprecipitated SMAD2 was visualized by in-gel fluorescence. (E) WT APT2 could remove the DHHC7-introduced S-palmitoylation on WT SMAD2 better than that on the amino acid 425-467 truncated mutant. DHHC7 knockout HEK293T cells were transfected with the indicated plasmids. The cells were labeled with Alk14 and the S-palmitoylation of SMAD2 was determined by in-gel fluorescence. (F) WT APT2 could remove the DHHC7-introduced S-palmitoylation on WT SMAD2 as well as C-terminal phosphorylation mutants. DHHC7 knockout HEK293T cells were transfected with the indicated plasmids. The cells were labeled with Alk14 and the S-palmitoylation of SMAD2 was determined by in-gel fluorescence and the p-SMAD2(C2) level was detected by western blot analyses. (G) DHHC7 knockout HEK293T cells were transfected with the indicated plasmids. SMAD2 was pulled down with Flag beads and subjected to western blot analyses.

FIGS. 8A-8E. DHHC7 promote linker phosphorylation (p-SMAD2(L3)) and activity of SMAD2. (A) Overexpression of Flag-SMAD2 WT, but not mutants, increased RORC mRNA level in HEK293T cells. (B) DHHC7 knockout HEK293T cells were transfected with the indicated plasmids. SMAD2 was pulled down with Flag beads and subjected to western blot analyses. (C) DHHC7 knockout HEK293T cells were transfected with the indicated plasmids. Cell lysate was subjected to western blot analyses. (D) Distribution of SMAD2 and p-SMAD2(L3) in different subcellular fractions of DHHC7 WT and knockout HEK293T cells (left). The relative SMAD2 and p-SMAD2(L3) level was quantified (right). (E) The subcellular localization of SMAD2 and p-SMAD2(L3) was analyzed using confocal imaging after Flag-tagged SMAD2 were transfected into DHHC7 WT and knockout HEK293T cells (left). The relative p-SMAD2(L3) level was quantified (right). Scale bars, 50 μm. The values are expressed as the means±SEM. **, P<0.01.

FIGS. 9A-9G. DHHC7 induced S-palmitoylation promote of the interaction of SMAD2 with SMAD4 and STAT3. (A), (B) Flag-SMAD4 pulled down HA-SMAD2 when co-expressed in HEK293T cells with indicated DHHC7 overexpression. The interaction of HA-SMAD2 with Flag-SMAD4 was much weaker in mutant DHHC7 overexpression compared with WT. The interaction of HA-SMAD2 C41/81S mutant with Flag-SMAD4 was much weaker. (C) Flag-STAT3 pulled down HA-SMAD2 when co-expressed with indicated DHHC7 in HEK293T cells. The interaction of HA-SMAD2 with Flag-STAT3 was much weaker in mutant DHHC7 than WT DHHC7. The interaction of Flag-STAT3 with HA-SMAD2 C41/81S mutant was much weaker than that with HA-SMAD2 WT. (D) Flag-SMAD2 pulled down HA-STAT3 when co-expressed with indicated DHHC7 in HEK293T cells. The interaction of HA-STAT3 with Flag-SMAD2 was much weaker in cells expressing mutant DHHC7 than WT DHHC7. The interaction of HA-STAT3 with Flag-SMAD2 C41/81S mutant was much weaker than that with Flag-SMAD2 WT. (E) The localization of Flag-SMAD2 and HA-STAT3 was analyzed using confocal imaging in WT and DHHC7 KO HEK293T cells (left). Scale bars were 50 μm. Colocalization of SMAD2 and STAT3 was quantified using Pearson's correlation coefficients (right). (F) WT and mutant Flag-SMAD2 pulled down of HA-STAT3 in HEK293T cells expressing DHHC7. The interaction of Flag-SMAD2 S255A mutant with HA-STAT3 was much weaker. (G) Co-overexpression of Flag-SMAD2 WT and HA-DHHC7 WT, but not mutants, increased RORC mRNA level in HEK293T cells. Quantification data are expressed as mean t SEM. Asterisks (*) indicate significant differences (**P<0.01).

FIGS. 10A-10F. The palmitoylation-depalmitoylation cycle of SMDA2 promotes Th17 cell differentiation. (A) DHHC7 promotes the linker phosphorylation SMAD2 in mouse splenocytes. SMAD2 and p-SMAD2(L3) blots are shown (left) and the relative p-SMAD2(L3) levels are quantified (right). DHHC7 also promoted the linker phosphorylation of SMAD2 C41/81S mutant, although to a much smaller extent. (B) Flow cytometry quantification of Th17 cell differentiation for samples used in (A). (C) APT2 has no significant effect on linker phosphorylation of either SMAD2 WT or C41/81S mutant in mouse splenocytes. SMAD2 and p-SMAD2(L3) blots are shown (left) and the relative p-SMAD2(L3) levels are quantified (right). (D) Flow cytometry quantification of Th17 cell differentiation for samples used in (C). (E) Flag-SMAD2 pull down of endogenous STAT3 in WT and Zdhhc7 knockout mouse splenocytes. The interaction of STAT3 with Flag-SMAD2 was much weaker in Zdhhc7 knockout splenocytes. Indicated blots are shown (left) and the relative STAT3/SMAD2 interaction levels are quantified (right). (F) Flag-SMAD2 pull down of endogenous STAT3 in WT and APT2 knockout mouse splenocytes. The interaction of STAT3 with Flag-SMAD2 was weaker in APT2 knockout splenocytes. Indicated blots are shown (left) and the relative STAT3-SMAD2 interaction levels are quantified (right). Quantification data are expressed as mean±SEM. *, P<0.05; **, P<0.01.

FIGS. 11A-11C. SMAD2 S-palmitoylation accelerates clinical score in MS mice models. WT, Zdhhc7 and Lypla2 knockout C57BL/6J mice were treated with MOG35-55 and pertussis toxin at the beginning, a second dose of pertussis toxin was treated on day two after immunization. Body weight changes (A), clinical score (B) and Th17 cell levels in spleen (C) were observed as indicated. Quantification data are expressed as mean±SEM. *, P<0.05; **, P<0.01.

FIGS. 12A-12D. 2-BP, a pan-DHHC inhibitor, decreases cytokines mRNA expression during the LPS priming step of inflammasome activation. Bone marrow-derived macrophages (BMDM) were treated with 100 ng/mL lipopolysaccharide (LPS), which can prime BMDM for inflammasome activation. A small molecule that inhibits all the DHHC family of palmitoyltransferases (DHHCs), 2-bromopalmitate (2-BP), was added at 10 μM or 25 μM together with LPS. The cells were cultured with LPS and 2-BP for 6 hours and then the mRNA levels of several pro-inflammatory cytokines: (A) IL-1 beta, (B) IL-6, (C) IL-12 beta, and (D) IL-18, were measured by quantitative reverse transcription PCR (qRT-PCR). 2-BP can decrease the mRNA levels of all these cytokines in a concentration dependent manner, suggesting that inhibiting DHHCs can decrease the priming step of inflammasome activation.

FIG. 13. 2-BP affects NLRP3-mediated inflammasome activation. Peritoneal macrophage was first primed with LPS (200 ng/mL) in DMEM medium (without serum) for 4h, and then 2-BP (25 μM) and ATP (5 mM) or Nigericin (10 μM) were added to the cell culture and incubated for 1 h. ATP and nigericin are two reagents that are commonly used to activate the NLRP3 inflammasome. The activation of NLRP3 inflammasome was then monitored by measuring the level of IL-1 beta secreted to the medium.

FIGS. 14A-14B. DHHC7 knockout decreases IL-1b and IL-18 Secretion in Bone-marrow-derived macrophage (BMDM) during inflammasome activation. DHHC7 WT and knockout BMDM were primed with LPS (10 ng/mL) in DEME medium overnight. The next day, the medium was change to DMEM with LPS (10 ng/mL) and ATP (5 mM), or DMEM with LPS (10 ng/mL) and nigericin (10 μM), and incubated for 1 h to activate the NLRP3 inflammasome. The medium was then collected and the secreted (A) IL-1 beta and (B) IL-18 were measured using ELISA kits. The results showed that DHHC7 knockout can significantly decrease NLRP3 inflammasome activity.

FIG. 15. APT2 knockout only has a slight effect on IL-1b Secretion in BMDM during inflammasome activation. APT2 WT and knockout BMDM were primed with LPS (200 ng/mL) in DEME medium for 4 hours. Then, the medium was change to DMEM with LPS (200 ng/mL) and ATP (5 mM), or DMEM with LPS (10 ng/mL) and nigericin (10 μM) and incubated for 1 h to activate the NLRP3 inflammasome. The medium was then collected, and the secreted IL-1 beta was measured using an ELISA kit. The results showed that APT2 knockout can also decrease NLRP3 inflammasome activity, although less so compared to DHHC7 knockout.

FIG. 16. DHHC7 knockout decreases inflammasome activation in mice: LPS-induced endotoxic shock. Adult (>8 weeks old) B6.129P2(FVB) DHHC7 WT or knockout mice were injected intraperitoneally with LPS (35 mg/kg) in about 100 μL of sterile PBS buffer. 12 hours later, the mice will be euthanized, and the blood was collected for analysis of IL-1beta levels in the serum. DHHC7 knockout significantly decreased the amount of IL-1beta secreted to the serum, suggesting that DHHC7 knockout decreased inflammasome activation in the mice.

FIGS. 17A-17B. Lupus Nephrosis Mouse Model: APT2 inhibitor ML349 decreases protein concentration in the mouse urine. (A) Protein concentration in urine (all data points), (B) Protein concentration in urine (averaged, with error bars). NZB/W F1 female mice at 25-week were administered via IP injection with either vehicle solution (DMSO+PBS) or APT2 inhibitor ML349 at 25 mg/Kg three times per week for 8 weeks. After 4 weeks of treatment, mice were evaluated for lupus incidence weekly through proteinuria analysis by collecting the urines and measuring the protein concentrations in the urine. The data showed that APT2 inhibitor ML349 can decrease the protein concentrations in the urine.

### DETAILED DESCRIPTION

The inventors of this disclosure have found that several proinflammatory transcription factors undergo a palmotylation/depalmotylation cycle, which is crucial for their proinflammatory effects. The inventors also found that disrupting the palmotylation/depalmotylation cycle of proinflammatory transcription factors is beneficial for treating inflammatory disorders.

### Methods for Treatment

In some embodiments, the method comprises administering to a patient suffering from an inflammatory disorder an effective amount of an inhibitor of an enzyme that regulates the S-palmitoylation of one or more pro-inflammatory transcription factor.

As used herein, the phrase “inflammatory disorder” refers to a disorder relating to, e.g., associated with or caused by, abnormal or unwanted inflammation. In some embodiments, the inflammatory disorder is selected from Inflammatory Bowel Disease (IBD) (e.g., Crohn's disease, ulcerative colitis), rheumatoid arthritis, vasculitis, pulmonary diseases (e.g., Chronic Obstructive Pulmonary Disease (COPD) and pulmonary interstitial disease (e.g., Idiopathic Pulmonary Fibrosis (IPF)), psoriasis, gout, allergic airway diseases (e.g., asthma, rhinitis), or endotoxic shock (e.g., from bloodstream infection with gram-negative bacteria, LPS-induced endotoxic shock). In some embodiments, the inflammatory disorder is an autoimmune disorder.

In some embodiments, an autoimmune disorder is selected from inflammatory bowel disease, multiple sclerosis, rheumatoid arthritis, lupus, graft versus host disease, type I diabetes, or psoriasis.

By “treating” a disorder is meant that the onset of the disorder is inhibited or delayed, the occurrence or frequency of the symptoms is inhibited or reduced, the progression of the disorder is slowed down, and/or the symptoms of the disorder are ameliorated.

As used herein the term “proinflammatory” refers to an inflammation-promoting effect. A proinflammatory transcription factor can be a transcription factor that directs transcription of a proinflammatory molecule such as, for example, a pro-inflammatory cytokine (e.g., an interleukin (IL) cytokine such as IL-1 beta, IL-6, IL-12 beta, IL-17 or IL-18), or a pro-inflammatory transcription factor such as ROR-γt. In some embodiments, a proinflammatory transcription factor is Signal Transducer and Activator of Transcription 3 (STAT3). In some embodiments, a proinflammatory transcription factor is Mothers against decapentaplegic homolog 2 (SMAD2). In some embodiments, STAT3 and/or SMAD2 positively regulate transcription of pro-inflammatory cytokines such as IL-1 beta, IL-6, IL-12 beta, IL-17 or IL-18, and lead to accelerated T_{H}17 cell differentiation.

In some embodiments, the enzyme that regulates the S-palmitoylation of a pro-inflammatory transcription factor is a Zinc finger DHHC-type palmitoyltransferase. In some embodiments, the enzyme is Zinc finger DHHC-type palmitoyltransferase 7 (ZDHHC7) (Genbank Gene IDs: 55625 (Homo sapiens), 102193 (Mus musculus)) or Zinc finger DHHC-type palmitoyltransferase 3 (ZDHHC3) (Genbank Gene IDs: 51304(Homo sapiens), 69035 (Mus musculus)). Human ZDHHC7 amino acid sequence is shown in SEQ ID NO: 1, human ZDHHC7 nucleotide sequence is shown in SEQ ID NO: 7. Mouse ZDHHC7 amino acid sequence is shown in SEQ ID NO: 2, mouse ZDHHC7 nucleotide sequence is shown in SEQ ID NO: 8. Human ZDHHC3 amino acid sequence is shown in SEQ ID NO: 3, human ZDHHC3 nucleotide sequence is shown in SEQ ID NO: 9. Mouse ZDHHC3 amino acid sequence is shown in SEQ ID NO: 4, mouse ZDHHC3 nucleotide sequence is shown in SEQ ID NO: 10. Human LYPLA2 amino acid sequence is shown in SEQ ID NO: 5, human LYPLA2 nucleotide sequence is shown in SEQ ID NO: 11. Mouse LYPLA2 amino acid sequence is shown in SEQ ID NO: 6, mouse LYPLA2 nucleotide sequence is shown in SEQ ID NO: 12.

In some embodiments, the enzyme is that regulates the S-palmitoylation of a pro-inflammatory transcription factor Lysophospholipase 2 (LYPLA2), also called acyl protein thioesterase 2 (APT2) (Genbank Gene IDs: 11313(Homo sapiens), 26394 (Mus musculus)).

In some embodiments, the inhibitors of this disclosure are administered alone or in combination with other drugs.

In some embodiments, the inhibitors of the instant disclosure are small molecule inhibitors. The term “small molecule” herein refers to small organic chemical compound, generally having a molecular weight of less than 2000 daltons, less than 1500 daltons, less than 1000 daltons, less than 800 daltons, or less than 600 daltons.

In some embodiments, an effective amount of a small molecule inhibitor is about 0.2 mg/kg to 100 mg/kg of the small molecule inhibitor. In other embodiments, the effective amount of a small molecule inhibitor is about 0.2 mg/kg, 0.5 mg/kg, 1 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg of the small molecule inhibitor. As used herein, the term “about” refers to ±10% of a given value.

In an embodiment, a small molecule inhibitor can be combined with a pharmaceutically acceptable carrier prior to administration. For the purposes of this disclosure, “pharmaceutically acceptable carriers” means any of the standard pharmaceutical carriers. Examples of suitable carriers are well known in the art and may include, but are not limited to, any of the standard pharmaceutical carriers such as a phosphate buffered saline solution and various wetting agents. Other carriers may include additives used in tablets, granules and capsules, and the like. Typically, such carriers contain excipients such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods.

A small molecule inhibitor can be admixed with a pharmaceutically acceptable carrier to make a pharmaceutical preparation in any conventional form including, inter alia, a solid form such as tablets, capsules (e.g., hard or soft gelatin capsules), pills, cachets, powders, granules, and the like; a liquid form such as solutions, suspensions; or in micronized powders, sprays, aerosols and the like.

In some embodiments, the small molecule inhibitor of the present disclosure can be administered by different routes of administration such as oral, oronasal, or parenteral route.

“Oral” or “peroral” administration refers to the introduction of a substance into a subject's body through or by way of the mouth and involves swallowing or transport through the oral mucosa (e.g., sublingual or buccal absorption) or both.

“Oronasal” administration refers to the introduction of a substance into a subject's body through or by way of the nose and the mouth, as would occur, for example, by placing one or more droplets in the nose. Oronasal administration involves transport processes associated with oral and intranasal administration.

“Parenteral administration” refers to the introduction of a substance into a subject's body through or by way of a route that does not include the digestive tract. Parenteral administration includes subcutaneous administration, intramuscular administration, transcutaneous administration, intradermal administration, intraperitoneal administration, intraocular administration, and intravenous administration.

In some embodiments, the enzyme is LYPLA2, and the small molecule inhibitor is ML349 with the following chemical formula:

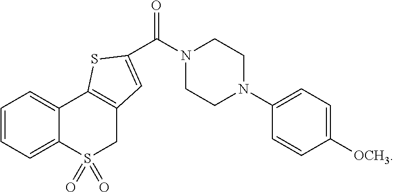 [SMILES: COc1ccc(N2CCN(C(=O)c3cc4c(s3)-c3ccccc3S(=O)(=O)C4)CC2)cc1]

In some embodiments, the enzyme is a Zinc finger DHHC-type palmitoyltransferase, and the small molecule inhibitor is 2-bromopalmitic acid 2-BP) with the following formula:

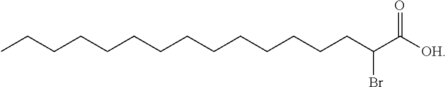 [SMILES: CCCCCCCCCCCCCCC(Br)C(=O)O]

In some embodiments, the enzyme is a Zinc finger DHHC-type palmitoyltransferase, and the small molecule inhibitor is cerulenin with the following formula:

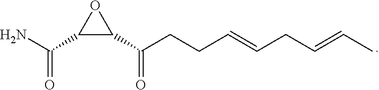

In some embodiments, the enzyme is a Zinc finger DHHC-type palmitoyltransferase, and the small molecule inhibitor is tunicamycin with the following formula:

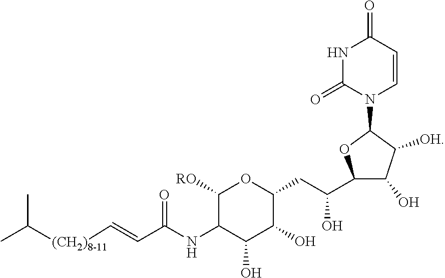

In some embodiments, the inhibitor of the enzyme is a nucleic acid inhibitor. A “nucleic acid inhibitor” is a nucleic acid that can reduce or prevent expression or activity of a target gene. For example, an inhibitor of expression of a Zinc finger DHHC-type palmitoyltransferase gene can reduce or eliminate transcription and/or translation of the Zinc finger DHHC-type palmitoyltransferase gene product, thus reducing Zinc finger DHHC-type palmitoyltransferase gene protein expression. For example, an inhibitor of expression of a Zinc finger DHHC-type palmitoyltransferase gene can reduce or eliminate transcription and/or translation of the Zinc finger DHHC-type palmitoyltransferase gene product, thus reducing Zinc finger DHHC-type palmitoyltransferase gene protein expression.

In some embodiments, the nucleic acid inhibitor is selected from the group consisting of an antisense RNA, a small interfering RNA, a microRNA, an artificial microRNA, and a ribozyme.

In some embodiments, the inhibitor of the enzyme is a genome editing system.

In some embodiments, the genome editing system is selected from the group consisting of CRISPR/Cas system, Cre/Lox system, TALEN system, ZFNs system and homologous recombination.

In some embodiments, the CRISPR-mediated genome editing comprises introducing into the patient a first nucleic acid encoding a Cas9 nuclease, a second nucleic acid comprising a guide RNA (gRNA), wherein said gRNA is specific to the gene encoding the enzyme.

### EXAMPLES

### Example 1: Materials and Methods

### Zdhhc7-Knockout Mice

The mouse strain used for this research project, B6.129P2(FVB)-Zdhhc7tm1.2Lusc/Mmmh, RRID:MMRRC_043511-MU, was obtained from the Mutant Mouse Resource and Research Center (MMRRC) at the University of Missouri, an NIH-funded strain repository, and was donated to the MMRRC by B. Luscher (The Pennsylvania State University). Genotype identification was performed according to the MMRRC protocol. Primers for the wild-type allele were as follows: forward: TGAGCCAGGATGGATTTCAGACA (SEQ ID NO: 13) and reverse: TGCCCTCGGACGCAGGAGATGAA (SEQ ID NO: 14). Primers for the mutant type allele were as follows: forward: TCCCCTGATGTATGCGAATGTCC (SEQ ID NO: 15) and reverse: AACAGGTGCCTITTGAATGTCAG (SEQ ID NO: 16).

### DSS-Induced Mouse Colitis Model

The mouse protocol 2019-0009 was approved by the Institutional Animal Care and Use Committee (IACUC) at Cornell University. All animals were housed under specific-pathogen-free conditions following the regulations of the IACUC. Mice (6-8 weeks old) were randomized into different groups (8 mice per group, mixed sex) as indicated. Colitis was induced by treating with 3.0% DSS (MP Biomedicals) in their drinking water ad libitum. ML349 solution was intraperitoneally injected into the mice at the indicated doses every other day. All mice were euthanized and the spleens were isolated to detect T cells. The distance from caecum to anus was measured. The colon was fixed in 4% paraformaldehyde for pathological examination. The study was not blinded.

### Common Reagents and Antibodies

The following reagents and antibodies were purchased from commercial sources: inhibitor cocktail (Trichostatin A (TSA, T8552, Sigma), protease inhibitor cocktail (P8340, Sigma), phosphatase inhibitor cocktail (P0044, Sigma)), fedratinib (S2736, Selleckchem), universal nuclease (88700, Thermo Fisher), Bradford assay (23200, Thermo Fisher), dithiothreitol (DTT; DTT100, Goldbio), enzyme-linked chemiluminescence (ECL) plus (32132, Thermo Fisher), SYBR Green PCR Master Mix (4472908, Applied Biosystems), streptavidin agarose (20359, Thermo Fisher), Protein A/G PLUS-Agarose (sc-2003, Santa Cruz Biotechnology), anti-Flag agarose gel (A2220, Sigma) and anti-HA affinity gel (E6779, Sigma). Antibodies were as follows: STAT3 (9139, CST), phospho-STAT3 (Tyr705) (ab76315, Abcam), β-actin (C4) HRP (SC-47778, Santa Cruz), Na/K-ATPase (SC-21712, Santa Cruz), histone H3 (4499S, CST), Flag HRP (A8592, Millipore), HA-probe (Y-11) (SC805, Santa Cruz), HA-probe (F-7) (SC7392, Santa Cruz), DHHC7 (ab138210, Abcam), DHHC7 (R12-3691, Assay Biotechnology), Alexa Fluor 350 goat anti-rabbit IgG (A-11046, Invitrogen), Alexa Fluor 594 goat anti-mouse IgG (8890S, CST), mouse CD4 PeiCP-Cy5.5 (560767, BD Pharmingen), mouse IL-17A PE (560767, BD Pharmingen), anti-mouse IgG HRP (7076S, CST) and anti-rabbit IgG HRP (7074S, CST).

### Cloning and Mutagenesis

APT2 and DHHC1-23 murine plasmids were provided by M. Fukata. DHHC3/7/19 human plasmids were obtained from GenScript. STAT3 expression vectors with different tags were obtained from Addgene. Point mutations of plasmids were generated by QuikChange site-directed mutagenesis.

### Cell Culture and Transfection

Human HEK293T cells (obtained from ATCC) were grown in DMEM media (11965-092, Gibco) with 10% bovine calf serum (CS, 12133C, Sigma) and extra 5% fetal bovine serum (FBS, 26140079, Gibco) to improve cell growth. ZDHHC7-knockout HEK293T cells were generated as previously described. In brief, design of the guide RNA (gRNA) was carried out using the CRISPR Design Tool (MIT) to minimize potential off-target effects. Three pairs of gRNA sequences (#1, 5′-caccgGAGGATGATGCTCGACGTCC-3′ (SEQ ID NO: 17), 5′-aaacGGACGTCGAGCATCATCCTCc-3′ (SEQ ID NO: 18); #2, 5′-caccgCGTCGAGCATCATCCTCTCC-3′ (SEQ ID NO: 19), 5′-aaacGGAGAGGATGATGCTCGACGc-3′ (SEQ ID NO: 20); #3, 5′-caccgCGGGTCTGGTTCATCCGTGA-3′ (SEQ ID NO: 21), 5′-aaacTCACGGATGAACCAGACCCGc-3′ (SEQ ID NO: 22)) were cloned in lentiCRISPR v2 vector (49535, Addgene) to generate ZDHHC7-targeting vectors. Then the targeting vector was transfected into HEK293T cells with FuGene 6 (E2691, Promega). The empty lentiCRISPR v2 vector was taken as control. Puromycin (2 μg ml⁻¹; P-600-100, GoldBio) was added in culture media after transfection for 24 h and cells were seeded as a single cell in each well of 96-well plates using a limited dilution method. Knockout of ZDHHC7 was confirmed by western blot and three independent strains of monoclonal ZDHHC7 knockout cell lines were selected for further experiments.

Splenocytes were isolated from mice by classic methods. In brief, the excised spleen was sliced into small pieces and placed onto a strainer (352350, Thermo Fisher) attached to a 50 ml conical tube. The sliced spleen was pressed through the strainer using the plunger end of a syringe and the cells were washed through the strainer with excess 4° C. PBS. The cell suspension was centrifuged at 500 g for 5 min at 4° C. The cell pellet was suspended in 2 ml of red blood cell lysing buffer (R7757, Sigma) for 5 min at RT and diluted with 30 ml PBS. The cells were centrifuged at 500 g for 5 min at RT. The cell pellet was suspended in 20 ml of 37° C. DMEM media, mixed well with 10 ml of Percoll density gradient media (17089102, VWR) and centrifuged at 2,500 g for 5 min at RT. The collected cells were seeded in 37° C. RPMI 1640 medium (12633012, Gibco) supplemented with 10% FBS at 5×10⁶ cells per ml. Splenocytes were cultured under T_{H}17-polarizing conditions: 3 ng ml⁻¹ TGF-β (100-21, PeproTech), 40 ng ml⁻¹ IL-6 (200-06, PeproTech), 30 ng ml⁻¹ IL-23 (200-23, PeproTech), 20 ng ml⁻¹ tumour necrosis factor (TNF) (300-01A, PeproTech) and 10 ng ml⁻¹ IL-1β (200-01B, PeproTech).

For HEK293T cells, the transient transfection was performed using FuGene 6 (E2691, Promega) or polyethylenimine (PEI) (24765, Polysciences). For splenocytes, the transient transfection was done using the Gene Pulser Xcell system with the recommended buffer (1652677, Bio-Rad) according to the manufacturer's protocol. LYPLA2 knockdown was performed with siRNA (136366, Thermo Fisher).

### Click Chemistry and In-Gel Fluorescence Detection

Cells were treated with 50 μM palmitic acid analogue Alkyne 14 (Alk14) for 5 h and the collected and lysed in 1% NP-40 lysis buffer (25 mM Tris-HCl pH 8.0, 150 mM NaCl, 10% glycerol, 1% Nonidet P-40) with protease inhibitor cocktail. The supernatant was collected after centrifugation at 16,000 g for 20 min at 4° C. The protein concentration was determined by Bradford assay (23200, Thermo Fisher). The target protein was purified with anti-Flag agarose beads and the beads were suspended in 50 μl of IP washing buffer. Click chemistry reagents were added to the beads in the following order: 1 μL of 4 mM TAMRA azide (47130, Lumiprobe), 1.2 μl of 10 mM tris[(1-benzyl-1H-1,2,3-triazol-4-yl)methyl]amine, (TBTA) (T2993, Tcichemicals), 1 μl of 40 mM CuSO₄, 1 μl of 40 mM tris(2-carboxyethyl)phosphine HCl (TCEP hydrochloride) (580560, Millipore). The reaction mixtures were mixed thoroughly and incubated for 30 min in the dark at room temperature. Then, 20 μl of 6×-SDS loading buffer was added and the resulting mixture was heated at 95° C. for 10 min. Half of the mixture was also treated with hydroxylamine (438227, Sigma) (pH 7.4, final concentration 500 μM) and heated for another 5 min at 95° C. to remove S-palmitoylation. The samples were then resolved by SDS-PAGE. For the overexpressed samples with high STAT3 levels, the gel was incubated with destaining buffer (50% CH₃OH, 40% water and 10% acetic acid) by shaking for 2-8 h at 4° C. and then incubated in water, which helped to lower the background. Otherwise, the gel was washed briefly in water. The gel was scanned to record the rhodamine fluorescence signal using a Typhoon 7000 Variable Mode Imager (GE Healthcare Life Sciences). After scanning, the gel was stained with Coomassie Brilliant Blue (B7920, Sigma) to check for protein loading.

### Acyl-Biotin Exchange

Acyl-biotin exchange (ABE) assays were performed as follows: Samples were suspended in 1 ml lysis buffer (100 mM Tris-HCl pH 7.2, 5 mM EDTA, 150 mM NaCl, 2.5% SDS, inhibitor cocktail) with 50 mM N-ethylmaleimide (NEM) (E3876, Sigma) and 50 U ml⁻¹ nuclease (88700, Thermo Fisher). Samples were solubilized at room temperature (RT) for 2 h with gentle mixing and centrifuged at 16,000 g for 20 min. The protein concentration of the supernatant was determined using a Bradford assay. Protein (2 μg) for each sample was precipitated with chloroform/methanol/water (v/v 1:4:3), briefly air-dried, and dissolved in 1 ml of lysis buffer with 5 mM biotin-HPDP (16459, Cayman Chemical) by gentle mixing at RT. Samples were then equally divided into two parts and incubated with 0.5 ml of 1 M hydroxylamine or negative control (1 M NaCl) respectively at RT for 3 h. Samples were precipitated again and dissolved in 200 μl of resuspension buffer (100 mM Tris-HCl pH 7.2, 2% SDS, 8 M urea, 5 mM EDTA). For each sample, 20 μl was used as loading control and 180 μl was diluted 1:10 with PBS and incubated with 20 μl of streptavidin beads with shaking overnight at 4° C. Beads were washed 3 times with PBS containing 1% SDS. The beads and loading controls were mixed with SDS loading buffer and heated at 95° C. for 10 min. Samples were then resolved by SDS-PAGE and subjected to western blot analyses.

### Western Blot

Cells were lysed with 1% NP40 lysis buffer and proteins were blotted following a standard protocol. Signals were detected using the chemiluminescence of ECL plus (32132, Thermo Fisher) on a Typhoon scanner.

### Subcellular Fractionation

Cells were collected and suspended in subcellular fraction buffer (250 mM sucrose, 20 mM HEPES, pH 7.4, 10 mM KCl, 1.5 mM MgCl₂, 1 mM EDTA, 1 mM EGTA and 1 mM DTT) containing protease inhibitor cocktail. Cells were homogenized with a 25-gauge syringe needle on ice. The lysate was centrifuged at 1,000 g for 5 min; the pellet was designated the nuclear fraction. The postnuclear supernatant was centrifuged at 6,000 g for 5 min to remove the mitochondrial fraction. The 6,000 g supernatant was subjected to centrifugation at 20,000 g for 2 h; the pellet was designated the membrane fraction. The 20,000 g supernatant was designated the cytosol fraction. All fractions were dissolved in 4% SDS lysis buffer (4% SDS, 50 mM triethanolamine pH 7.4 and 150 mM NaCl). Equivalent portions of different fractions were then subjected to western blot analyses.

### Immunofluorescence

Cells were seeded in 35-mm glass bottom dishes (MatTek) and fixed with 4% paraformaldehyde (v/v in PBS) for 30 min. The fixed cells were washed twice with PBS, permeabilized and blocked with 0.1% saponin/5% BSA/PBS for 30 min. The permeabilized cells were incubated overnight at 4° C. in the dark with primary antibody, followed by incubation with secondary antibody at RT in the dark for 1 h. Samples were mounted with Fluoromount-G (0100-01, SouthemBiotech) or DAPI Fluoromount-G (0100-20, SouthemBiotech) and observed using inverted confocal microscopy (LSM880, Zeiss).

qPCR

For the gene expression analysis, the qPCR was performed using SYBR Green PCR Master Mix according to the manufacturer's standard protocol.

### Flow Cytometry Analysis

For FACS analysis, flow cytometry was performed using 1×10⁶ cells per sample. The T_{H}17 cells were stimulated with cytokines cocktail: 3 ng ml⁻¹ TGF-β (100-21, PeproTech), 40 ng ml⁻¹ IL-6 (200-06, PeproTech), 30 ng ml⁻¹ IL-23 (200-23, PeproTech), 20 ng ml⁻¹ TNF (300-01A, PeproTech) and 10 ng ml⁻¹ IL-1β (200-01B, PeproTech), and then labelled with Cy5.5-CD4 (560767, BD Pharmingen). After permeabilization and fixation, the cells were labelled with PE-IL-17 (560767, BD Pharmingen). The cells were detected by Attune Flow Cytometer (Thermo Fisher) and analysed with FCS Express 6 software (De Novo Software).

### Statistical Analysis

Quantitative analyses were performed with SPSS 17.0 and data was expressed as mean t s.e.m. Comparisons among groups were performed using Student's t-test and other data were analyzed using a one-way analysis of variance (ANOVA).

### Example 2

STAT3 is palmitoylated by DHHC7

Recruitment of STAT3 to the plasma membrane is essential for its phosphorylation. The inventors first inquired whether S-palmitoylation would contribute to the membrane association of STAT3.

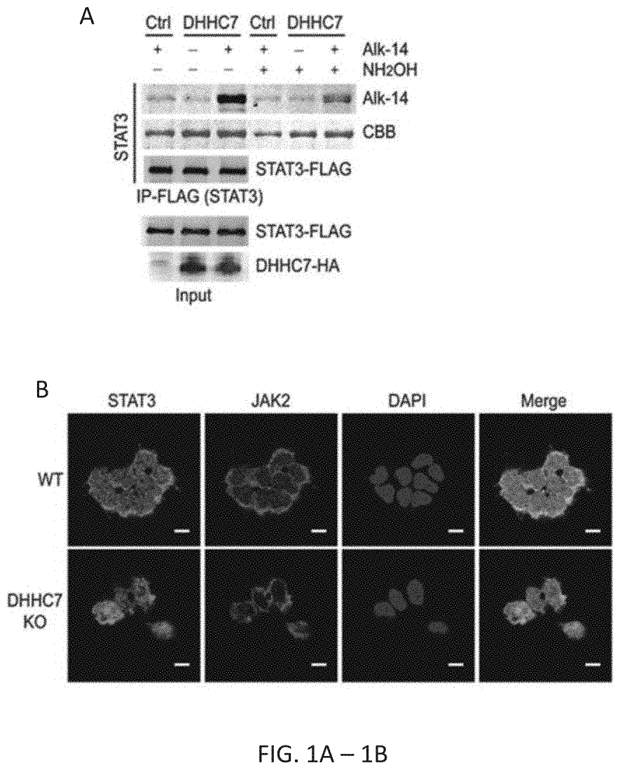

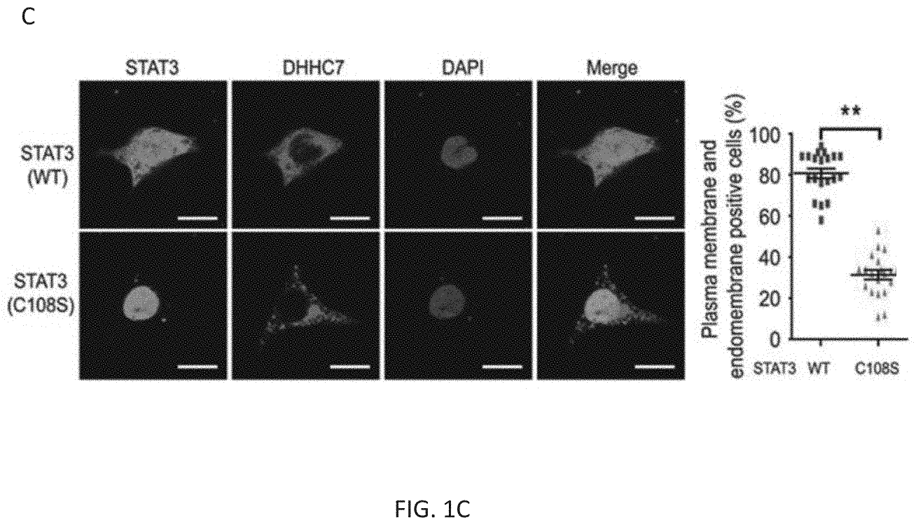

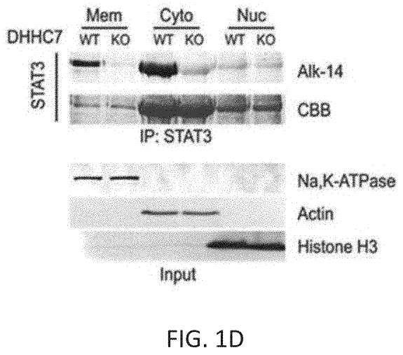

In order to visualize STAT3 palmitoylation in the presence of mouse DHHCs in HEK293T cells, the inventors used the alkyne-tagged palmitic acid analogue Alk14 as a metabolic label, which can conjugate with the fluorescent dye TAMRA azide via click chemistry. DHHC7 (encoded by Zdhhc7) and DHHC3 (encoded by Zdhhc3) increased the palmitoylation level of STAT3. Quantification revealed that STAT3 palmitoylation increased 5.4-fold upon DHHC7 expression; the effect of DHHC3 was weaker. Palmitoylation can occur on cysteine or lysine residues (on sulfur or nitrogen, respectively), but only S-palmitoylation is sensitive to hydroxylamine. Treatment with hydroxylamine removed more than 90% of the palmitoylation signal on STAT3, suggesting that palmitoylation of STAT3 by DHHC7 primarily occurred on cysteine (FIG. 1A).

To confirm that DHHC7 is the endogenous STAT3 palmitoyltransferase, the inventors generated DHHC7-knockout HEK293T cells and mouse splenocytes. S-palmitoylation of STAT3 in DHHC7-knockout cells was significantly decreased compared with control cells. Re-expression of wild-type DHHC7—but not of the catalytically inactive DHHS7 mutant, containing a cysteine-to-serine substitution in the conserved motif—significantly increased STAT3 palmitoylation. The inventors further confirmed DHHC7-promoted STAT3 palmitoylation using an acyl-biotin exchange assay, another commonly used method to detect S-palmitoylation.

Given that human DHHC19 was reported to act as the palmitoyltransferase for STAT3, the inventors considered whether sequence differences between human and mouse DHHCs could account for the difference in findings. The inventors tested human DHHC3, DHHC7 and DHHC19, and found that human DHHC7 is the most efficient STAT3 palmitoyltransferase.

There are seven members in the STAT family, of which STAT1 is the most similar to STAT3. Using an acyl-biotin exchange assay, the inventors showed that STAT1 is also palmitoylated; however, its palmitoylation levels were not increased in the presence of DHHC7.

### Palmitoylation Targets STAT3 to Membranes

To map the palmitoylation site of STAT3, the inventors individually mutated each of the 14 cysteine residues of STAT3 to serine and examined the palmitoylation status of the mutants. The palmitoylation signal of STAT3 showed a notable decrease only when Cys108 was mutated. Notably, the interaction between STAT3(C108S) and DHHC7 also decreased compared with that involving wild-type STAT3. Overexpression of neither DHHC7 nor DHHC3 could increase the palmitoylation of STAT3(C108S).

Because S-palmitoylation can target proteins to membranes, and STAT3 needs to be recruited to the plasma membrane in order to interact with JAK2, the inventors next examined whether S-palmitoylation affects STAT3 membrane recruitment. Wild-type STAT3 was localized at the plasma membrane, on endomembranes, and in the nucleus. Knockout of DHHC7 in HEK293T cells decreased the membrane localization of STAT3 but increased its nuclear localization (FIG. 1B). Consistent with this, STAT3(C108S) was found prominently in the nucleus, suggesting that palmitoylation promotes the membrane localization of STAT3. Furthermore, the re-expression of DHHC7 in DHHC7-knockout HEK293T cells led to the membrane recruitment of wild-type STAT3 but not of STAT3(C108S) (FIG. 1C). In both HEK293T cells and mouse splenocytes, DHHC7 induced palmitoylation of STAT3 and increased the amount of the modified protein in the membrane but not in the nuclear fractions (FIG. 1D). Endogenous STAT3 co-localized to a greater extent with JAK2 in wild-type than in DHHC7-knockout cells (FIG. 1B). Collectively, the results suggest that DHHC7-catalysed palmitoylation promotes the membrane localization of STAT3 and its interaction with JAK2.

### DHHC7 Promotes STAT3 Activation

The transcriptional activity of STAT3 is dependent on its phosphorylation at Y705. The inventors next examined whether palmitoylation could facilitate STAT3 phosphorylation. Consistent with the palmitoylation screen, STAT3 phosphorylation was notably (and selectively) increased by the expression of DHHC7 or DHHC3, with DHHC7 being the more effective. Endogenous STAT3 phosphorylation was regulated similarly by DHHC7 expression in both HEK293T cells and mouse splenocytes. DHHC7 knockout in HEK293T cells decreased the phosphorylation of endogenous wild-type STAT3, but not of the ectopically expressed mutant STAT3(C108S). The inventors therefore concluded that DHHC7 regulates STAT3 phosphorylation.

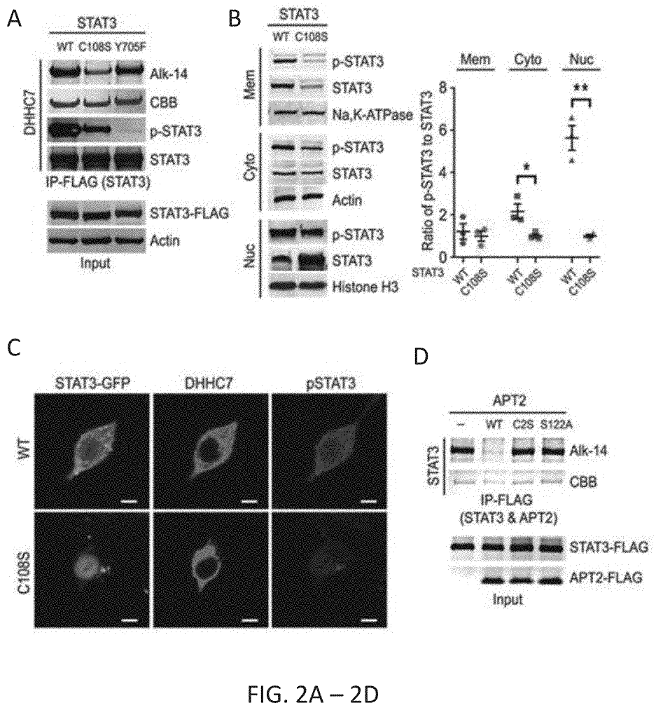

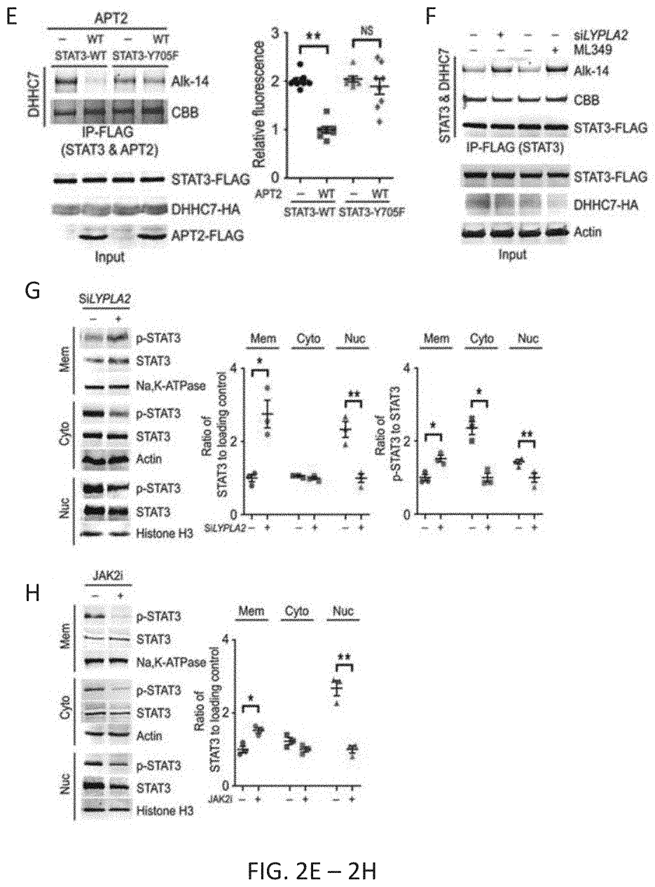

The inventors next co-expressed DHHC7 with wild-type STAT3, STAT3(C108S) and STAT3(Y705F). Phosphorylation of STAT3(C108S) was reduced relative to that of wild-type STAT3, whereas the palmitoylation status of STAT3 was unaffected by mutation of the Y705 phosphorylation site (FIG. 2A). Thus, STAT3 phosphorylation is facilitated by palmitoylation, but phosphorylation does not affect palmitoylation by DHHC7.

Subcellular fractionation showed that DHHC7 increased the membrane recruitment of STAT3 as well as the p-STAT3 signal located on membranes and in the nucleus. Notably, the ratio of p-STAT3 to STAT3 was increased by DHHC7 only in the nuclear fraction. The membrane recruitment and phosphorylation of STAT3(C108S) was reduced relative to that of wild-type STAT3 (FIG. 2B). Similarly, immunofluorescence imaging showed that, in DHHC7-expressing cells, wild-type STAT3 was more extensively located at the plasma membrane and endomembrane and had higher phosphorylation levels compared with STAT3(C108S), which was localized mainly in the nucleus (FIG. 2C). These data additionally support that the palmitoylation of STAT3 facilitates its phosphorylation by promoting its recruitment to the membrane, where JAK2 kinase is localized.

APT2 Depahnitoylates p-STAT3

APTs are involved in regulating the membrane localization of target proteins by depalmitoylation. Both APT1 and APT2 can be palmitoylated on Cys2, which promotes their membrane localization and access to substrates in membranes. It has recently been shown that APT1 is mainly localized in the mitochondria. The inventors therefore focused on testing whether STAT3 is a substrate of APT2. Haemagglutinin (HA)-tagged STAT3 and Flag-tagged APT2 were found to associate with each other in HEK293T cells. Wild-type APT2 interacted with STAT3 more strongly than did the mutant APT2(C2S). Expression of APT2 decreased the STAT3 palmitoylation signal (FIG. 2D). The C2S mutant or the catalytically inactive S122A mutant of APT2 failed to decrease the palmitoylation signal of STAT3 (FIG. 2D). APT2 knockdown and pharmacological inhibition of APT2 with ML349 also increased STAT3 palmitoylation (FIG. 2F). These results indicate that APT2 can depalmitoylate STAT3.

The inventors next evaluated whether depalmitoylation by APT2 regulates STAT3 phosphorylation and transcriptional activity. Given that DHHC7-catalysed palmitoylation promotes STAT3 activity, the inventors expected that STAT3 activity would be increased upon APT2 knockdown. However, APT2 knockdown inhibited both the transcriptional activity of STAT3 and its nuclear translocation (FIG. 2G). Furthermore, co-expression of wild-type DHHC7 and APT2 promoted the expression of downstream genes to a greater extent than their mutant counterparts. The dimerization of STAT3 is reported to be important for its transcriptional activity and is regulated by various post-translational modifications; however, it was not affected by palmitoylation.

To explain the finding that both palmitoylation and depalmitoylation promote STAT3 signalling, the inventors suggest that depalmitoylation of STAT3 occurs mainly on p-STAT3-serving to release p-STAT3 from membranes to promote its nuclear translocation. To test this hypothesis, the inventors generated the HA-tagged phosphorylation site mutant STAT3(Y705F) and repeated the STAT3-APT2 interaction experiment. Compared with wild-type STAT3, the association between STAT3(Y705F) and APT2 was reduced. Consistent with the results of the physical interaction studies, depalmitoylation of STAT3(Y705F) by APT2 was much less efficient (FIG. 2E). When JAK2 was inhibited with fedratinib, phosphorylation of STAT3 decreased as expected; however, levels of membrane-localized STAT3 increased whereas those of nuclear STAT3 decreased (FIG. 2H). Collectively, the data support the hypothesis that APT2 preferentially promotes the depalmitoylation and nuclear translocation of p-STAT3 over STAT3.

### Example 3

### STAT3 Palmitoylation Cycle Promotes T_{H}17

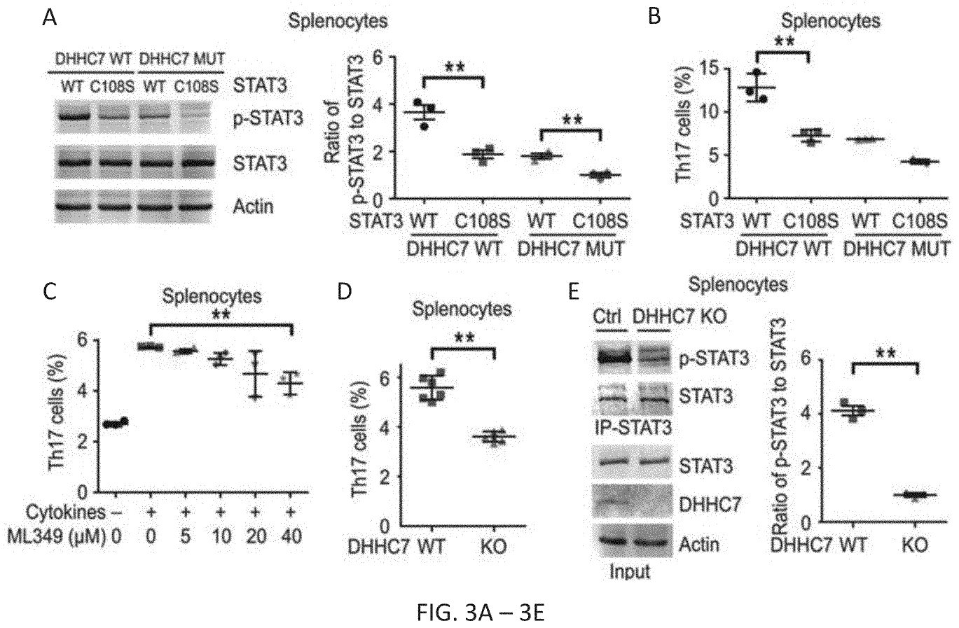

Because STAT3 is important for T_{H}17 cell differentiation, the inventors evaluated whether the palmitoylation-depalmitoylation cycle of STAT3 promotes the generation of T_{H}17 cells from mouse spleen cells. Under T_{H}17 differentiation conditions, STAT3 phosphorylation and transcriptional activity were promoted to a greater extent when STAT3 was co-expressed with wild-type DHHC7 than with inactive DHHS7 (FIG. 3A). These results were further confirmed by quantification of T_{H}17 cells using flow cytometry (FIG. 3B). Inhibition of APT2 by ML349 significantly decreased the expression of STAT3 target genes (RORC and CCND1) and the differentiation of T_{H}17 cells (FIG. 3C). Zdhhc7 knockout also decreased T_{H}17 cell differentiation (FIG. 3D) and STAT3 phosphorylation (FIG. 3E). The palmitoylation-depalmitoylation cycle is therefore important for STAT3 signalling and T_{H}17 differentiation.

DHHC7 and APT2 are Upregulated in Patients with IBD

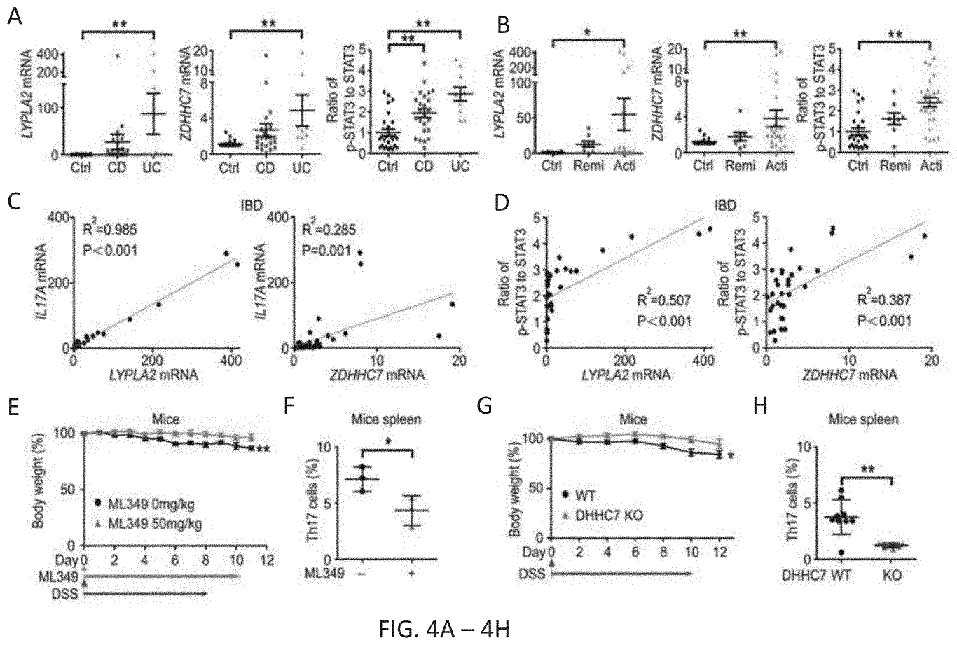

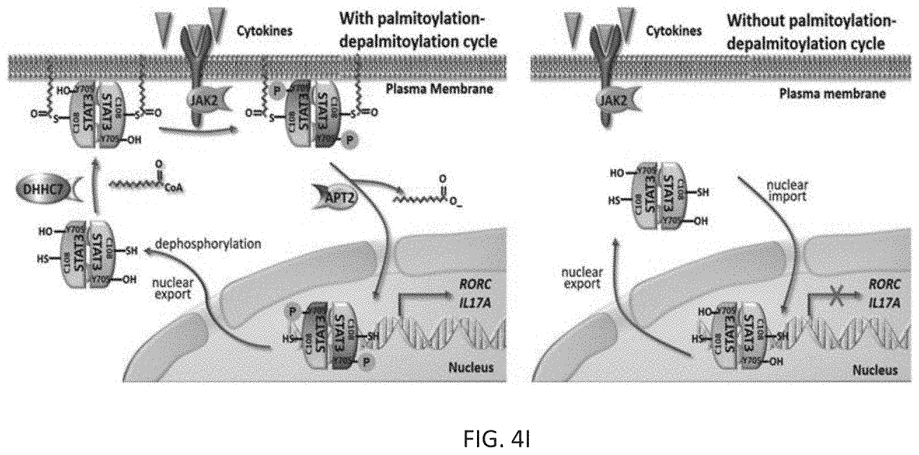

Activated STAT3 is suggestive of a poor prognosis in various autoimmune diseases, and the level of T_{H}17 cells is a key factor that affects the course and severity of intestinal inflammation. To determine whether expression levels of ZDHHC7 and LYPLA2—genes that promote the palmitoylation-depalmitoylation cycle—are correlated with intestinal inflammation in humans, human peripheral blood mononuclear cells (PBMCs) from 26 healthy participants, 24 patients with Crohn's disease and 10 patients with ulcerative colitis were extracted and analysed. ZDHHC7 and LYPLA2 mRNA was upregulated in patients with IBD, especially those with ulcerative colitis (FIG. 4A). Downstream target genes of STAT3—RORC and IL17A—were also highly expressed. In addition, cells from individuals with more active IBD show higher expression levels of ZDHHC7, LYPLA2, RORC and IL17A (FIG. 4B). There was a significant correlation between the expression of the STAT3 target genes (RORC and IL17A) and that of ZDHHC7 and LYPLA2 (FIG. 4C). Furthermore, p-STAT3 levels correlated with levels of ZDHHC7 and LYPLA2 mRNA in PBMCs (FIG. 4D). Notably, ZDHHC7 mRNA levels correlated with those of STAT3 target genes—as well as levels of p-STAT3—only in patients with IBD, whereas mRNA levels of LYPLA2 and STAT3 target genes showed excellent correlation in both healthy participants and in patients with IBD (FIGS. 4C and 4D). These results suggest that changes in the expression of LYPLA2 might be more relevant for IBD. The expression of ZDHHC7 correlated less well with that of STAT3 target genes—probably because DHHC3 could also regulate STAT3, as the data above indicates. Consistent with this hypothesis, ZDHHC3 expression levels were also increased in patients with IBD compared with healthy participants.

### Targeting APT2 or DHHC7 Reduces Colitis in Mice

The inventors tested whether the pharmacological inhibition of APT2 with ML349 could reduce dextran sulfate sodium (DSS)-induced colitis in mice, an experimental model for IBD. ML349 (50 mg kg⁻¹) was well tolerated by the mice. In the DSS-induced mouse model of colitis, pretreatment with ML349 followed by treatment with DSS significantly attenuated weight loss and increased the survival rate—indicating that ML349 treatment could effectively prevent DSS-induced colitis. DSS treatment followed by ML349 treatment also significantly attenuated weight loss and colon shortening in mice (FIG. 4E), indicating that ML349 could alleviate DSS-induced colitis. Furthermore, consistent with the in vitro results (FIG. 3), ML349 significantly decreased the levels of T_{H}17 cells in mouse splenocytes (FIG. 4F).

To provide additional support for these findings, the inventors also used Zdhhc7-knockout mice in the colitis model. On the basis of the mechanistic model (FIG. 4I), the inventors predicted that knockout of Zdhhc7 should also reduce DSS-induced colitis. Indeed, the inventors found that Zdhhc7 knockout decreased T_{H}17 cell differentiation and protected mice from DSS-induced colitis (FIGS. 4G and 4H). The inventors therefore suggest that the STAT3 palmitoylation-depalmitoylation cycle could be a promising therapeutic target for T_{H}17-related immune disorders.

The transcription factor STAT3 is known to be recruited to the plasma membrane and phosphorylated by JAK2 under specific stimulation. p-STAT3 then migrates to the nucleus and promotes the expression of target genes. However, very little is known about the mechanism by which STAT3 is recruited to the membrane. Here, the inventors showed that Cys108 of STAT3 is palmitoylated by DHHC7 (and to a lesser extent by DHHC3), which promotes membrane recruitment and phosphorylation by JAK2. Although palmitoylation is well known to be important for membrane distribution and signalling outputs, how palmitoylation and depalmitoyation are balanced to promote signalling has been a fundamental unaddressed question. Palmitoylation anchors STAT3 to the cell membranes, but for nuclear translocation, it must be depalmitoylated. The inventors showed that APT2 contributes to the nuclear translocation of p-STAT3 by selectively depalmitoylating p-STAT3 over unphosphorylated STAT3. These results suggest a model in which the palmitoylation-depalmitoylation cycle, rather than being a futile cycle, drives STAT3 activation (FIG. 4I). Without this cycle, even though STAT3 can still form homodimers and translocate to the nucleus, most STAT3 is present in its inactive unphosphorylated state (FIG. 4I).

Constitutive activation of STAT3 contributes to T_{H}17 differentiation in patients with immune disorders, leading to poor clinical outcomes. STAT3 has been proven to be an effective target for inhibiting T_{H}17 cell differentiation and attenuating colitis in mouse models of IBD. The present work demonstrates that the palmitoylation-depalmitoylation cycle of STAT3 affects T_{H}17 cell differentiation and suggests that both DHHC7 and APT2 could be new therapeutic targets for treating colitis. Because T_{H}17 cells are a key factor affecting the course and severity of various immune disorders such as IBD, hyper-IgE syndrome and arthritis, the palmitoylation-depalmitoylation cycle of STAT3 could be a potential therapeutic target for the treatment of many other autoimmune disorders.

Post-translational modifications are particularly suited for mediating cell signalling, as evidenced by the well-known signalling functions of phosphorylation and ubiquitination. Protein S-palmitoylation was discovered as a post-translational modification several decades ago, yet despite the fact that close to 3,000 proteins in humans are known to undergo this modification, very little is understood about how it contributes to cell signalling. This study demonstrates that a palmitoylation-depalmitoylation cycle can proceed in a specific direction to promote cell signalling, with the direction of the cycle in this case ensured by the specificity of APT2 towards the phosphorylated substrate. This example could provide important insights for understanding the signalling functions of S-palmitoylation in numerous other cell signalling processes.

### Example 4

### SMADs is Palmitoylated by DHHC7

In the R-Smads family, there are six members, including SMAD1, SMAD2, SMAD3, SMAD5 and SMAD8/9, which play important roles in the direct signal transduction of TGFβR and the differentiation of T cells, with SMAD2 being most similar to SMAD3. Previous studies have found that the key to phosphorylate inactive R-Smads is to recruit SMADs to the plasma membrane and cytoplasm where TGFβR and ERK are located. The inventors wondered whether S-palmitoylation would play a role in R-SMADs membrane association, similar to the regulation of STAT3 by S-palmitoylation. So far, no S-palmitoylation of SMAD2 and SMAD3 has been reported based on the database on protein S-palmitoylation, SwissPalm.

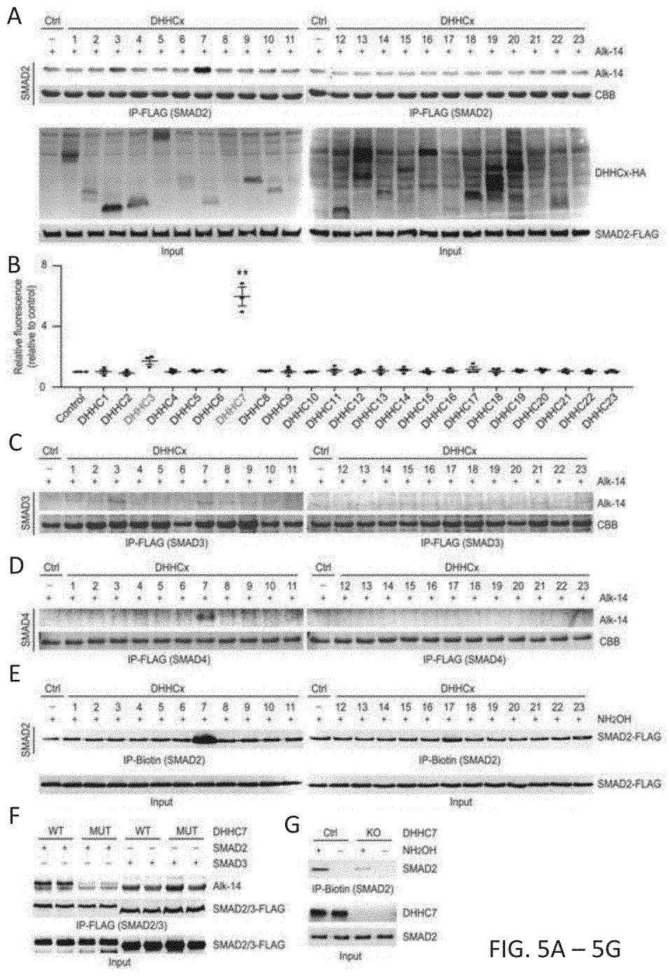

The inventors used metabolic labeling with metabolic Alk14, an alkyne-tagged palmitic acid analog, followed by TAMRA-azide conjugation via click chemistry and in-gel fluorescence to detect SMAD2/3 palmitoylation. The inventors found that SMAD2 can be labeled by Alk14 and the labeling can be significantly increased by DHHC7 (gene name Zdhhc7) expression (FIGS. 5A-5B). Quantification of the palmitoylation signal showed that SMAD2 palmitoylation was increased six-fold by DHHC7 (FIG. 5B). DHHC3 also increased SMAD2 palmitoylation but the change was not significant (FIG. 5B). In contrast, SMAD3 and SMAD4 had essentially undetectable Alk14 labeling and even in the presence of DHHC7 overexpression, the labeling remained very weak (FIGS. 5C, 5D and 5F). Acyl-biotin exchange (ABE) assay was also used to visualize the palmitoylation signal of SMAD2 in HEK 293T cells expressing different mouse DHHCs. Consistent with the Alk14 labeling data, only DHHC7 significantly promoted SMAD2 palmitoylation (FIG. 5E). To further determine if DHHC7 is an endogenous SMAD2 palmitoyltransferase, DHHC7 was knocked out in HEK 293T cells. The S-palmitoylation of SMAD2 was significantly reduced by DHHC7 knockout based on the ABE assay (FIG. 5G).

### Cys41 and Cys81 Palmitoylation Promotes the Membrane Recruitment of SMAD2

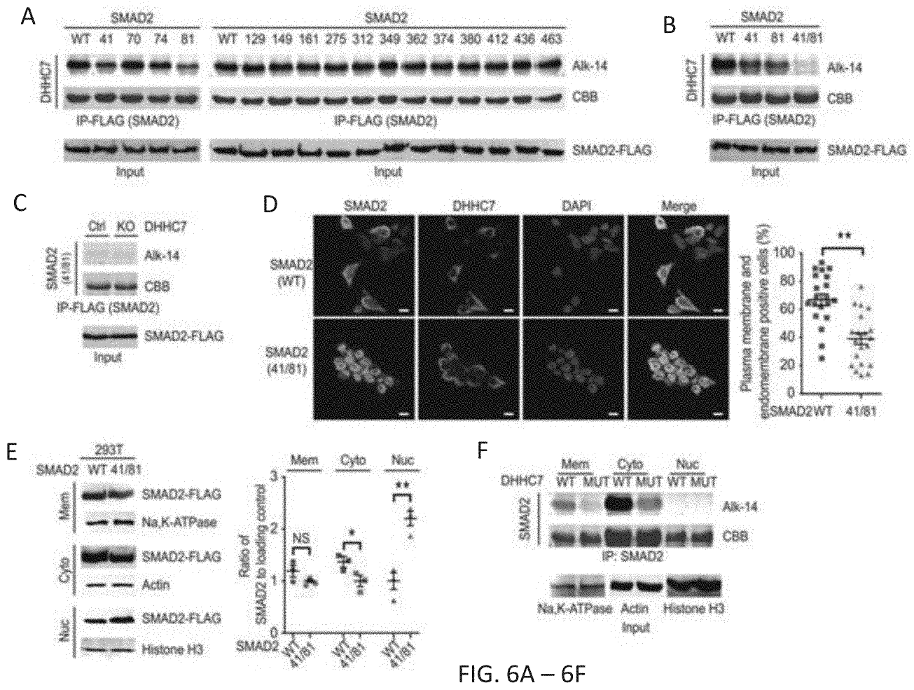

To identify the SMAD2 S-palmitoylation site, each of the 15 cysteine residues of SMAD2 was mutated to serine, and the S-palmitoylation status of each mutant was determined by Alk14 labeling. The inventors found that the Cys41 and Cys81 mutants significantly reduced the SMAD2 S-palmitoylation signal and Cys41/81 double mutant had almost no palmitoylation signal (FIGS. 6A-6B). DHHC7 knockout did not reduce the S-palmitoylation of the SMAD2 Cys41/81Ser mutant (FIG. 6C), suggesting that DHHC7-catalyzed SMAD2 palmitoylation occurs mainly on these two Cys residues.

S-palmitoylation can target proteins to cellular membranes, while SMAD2 needs to be recruited to the plasma membrane and cytoplasm to interact with TGFβR and ERK respectively. The inventors studied the effect of S-palmitoylation on SMAD2 localization. The inventors found that WT SMAD2 located in the plasma membrane, cytoplasm (likely membrane organelles in the cytoplasm), and nucleus, whereas the C41/81S mutant of SMAD2 is mainly located in the nucleus (FIGS. 6D-6E). Moreover, DHHC7 increased the S-palmitoylation of SMAD2 in the membrane and cytoplasm but not in the nuclear fractions (FIG. 6F), further supporting that palmitoylation promotes the membrane and cytoplasm localization of SMAD2. DHHC7 knockout in HEK293T cells also reduced the membrane localization and increased the nuclear localization of SMAD2. These findings show that DHHC7-catalyzed S-palmitoylation promotes SMAD2 membrane localization.

S-Palmitoylation does not Affect SMAD2 C-Terminal Phosphorylation

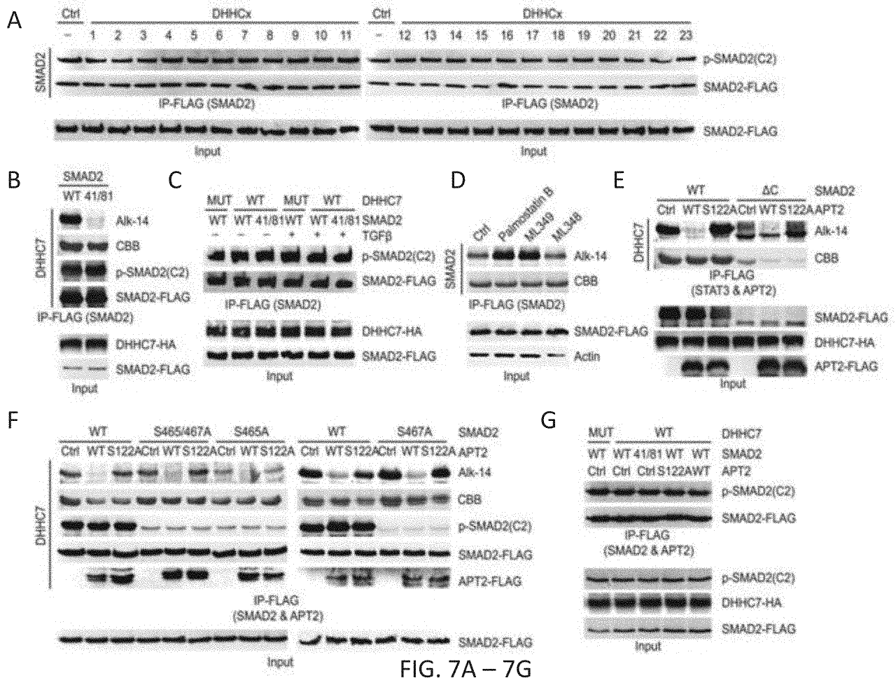

The transcriptional activity of SMAD2 and SMAD3 is activated by the phosphorylation at C-terminal (Ser465/Ser467 for SMAD2 and Ser423/Ser425 for SMAD3). Above, in Example 2, the inventors demonstrate that DHHC7-induced S-palmitoylation can promote the phosphorylation of another transcription factor STAT3 and promote its transcriptional activity. Thus, the inventors hypothesized that the palmitoylation of SMAD2 may had a similar effect with palmitoylation promoting the C-terminal Ser465/Ser467 phosphorylation of SMAD2 and activate its transcriptional activity. The inventors examined whether co-expressing the HA-tagged DHHC proteins and FLAG-tagged SMAD2 in HEK-293T cells would increase SMAD2 C-terminal Ser465/Ser467 phosphorylation (p-SMAD2(C2)). Surprisingly, DHHC7 did not promote p-SMAD2(C2) levels (FIG. 7A). Furthermore, both SMAD2 WT and the S-palmitoylation-deficient C41/81S mutant had similar levels of p-SMAD2(C2) under DHHC7 expression and TGFβ treatment (FIGS. 7B-7C). Therefore, the DHHC7-induced S-palmitoylation of SMAD2 does not affect p-SMAD2(C2) level.

### SMAD2 S-Palmitoylation is Removed by APT2

S-Palmitoylation can be removed by acyl protein thioesterases (APT1, APT2, and ABHD family members). APT2 (LYPLA2, Lysophospholipase 2) is involved in regulating the membrane localization of STAT3 and prefers p-STAT3 over unphosphorylated STAT3 as a depalmitoylation substrate. The inventors examined which acyl protein thioesterases regulates the SMAD2 S-Palmitoylation. HEK-293T cells expressing Flag-tagged SMAD2 were treated with palmostatin B (a pan-depalmitoylase inhibitor), ML348 (an APT1-specific inhibitor) or ML349 (an APT2-specific inhibitor). The inventors found that both ML349 and palmostatin B, but not ML348, significantly enhanced the S-palmitoylation signal, suggesting that APT2 could depalmitoylate SMAD2 (FIG. 7D). To further confirm this result, the inventors used the Ser22Ala (S122A) catalytically dead mutant of APT2 and found that S122A mutant APT2 failed to reduce the S-palmitoylation signal of SMAD2 (FIGS. 7E-7F). These data suggested that APT2 can depalmitoylate SMAD2.

Subsequently, the inventors evaluated whether APT2 prefers p-SMAD2 or unphosphorylated SMAD2, similar to the case of STAT3. The inventors first used the carboxy-terminal truncated SMAD2 (AC SMAD2, truncated from Glu425 to Ser467) and found that compared with WT SMAD2, AC SMAD2 was largely resistant to the depalmitoylation effect of APT2 (FIG. 7E). This suggested that the depalmitoylation by APT2 is dependent on the C-terminal of SMAD2. However, unlike the preference for p-STAT3, APT2 did not show preference for p-SMAD2(C2) over unphosphorylated SMAD2, as mutating either Ser465, Ser467, or both did not affect the depalmitoylation by APT2 (FIG. 7F). Moreover, APT2 did not significantly affect the phosphorylation of SMAD2 on Ser465/Ser467 (FIG. 7G). Thus, although DHHC7 and APT2 controls the S-palmitoylation of SMAD2, they do not affect SMAD2 phosphorylation at the C-terminal serines.

### DHHC7-Induced S-Palmitoylation Promotes SMAD2 Phosphorylation at the Linker Region.

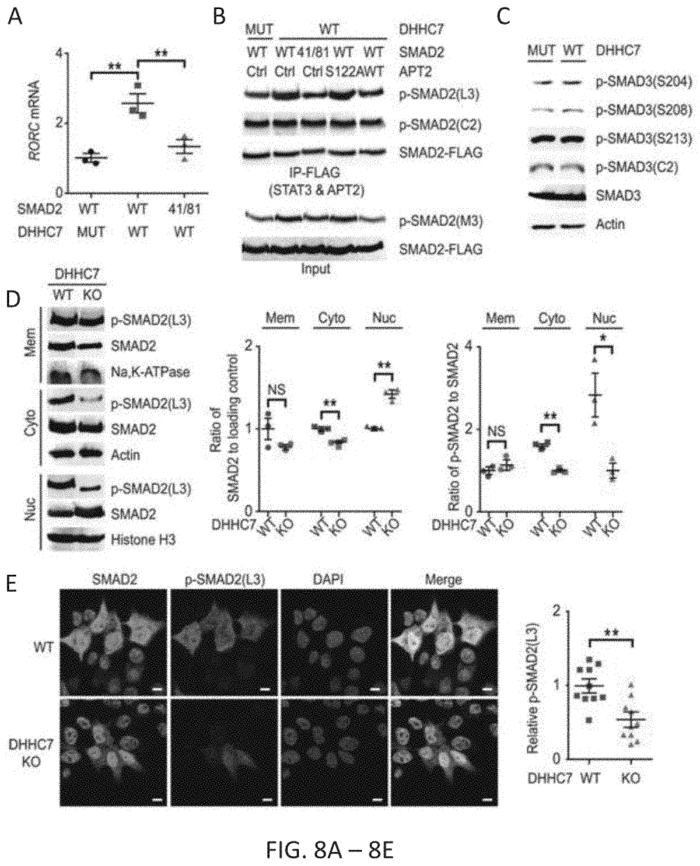

To find out whether DHHC7-induced S-palmitoylation promotes the transcriptional function of SMAD2, the inventors evaluated the downstream gene expression by real time PCR. The inventors found that co-expression of WT DHHC7 and SMAD2 induced higher RORC expression than the co-expression of inactive mutant DHHC7 and SMAD2, or the co-expression of WT DHHC7 and S-palmitoylation deficient SMAD2 C41/81S mutant (FIG. 8A). These results strongly support that DHHC7-catalyzed SMAD2 S-palmitoylation does affect its transcriptional activity, despite the similar C-terminal phosphorylation level on SMAD2.

SMAD2 and SMAD3 are composed of an amino-terminal (N-terminal) Mad homology-1 (MH1) domain, a linker region and a C-terminal MH2 domain. Phosphorylation at linker region (Ser245/250/255 for SMAD2 and Ser204/208/213 for SMAD3) are also important for the transcriptional activity of R-SMADs. The inventors examined whether the phosphorylation of SMAD2 at linker region (p-SMAD2(L3)) could be regulated by palmitoylation. Interestingly, different from the C-terminal phosphorylation, the linker region phosphorylation of SMAD2 was significantly increased by DHHC7, as SMAD2 C41/81S mutant or APT2-induced depalmitoylation decreased p-SMAD2 (L3) (FIG. 8B). Although SMAD3 is very similar to SMAD2 and has the same linker region domain, it is not palmitoylated (FIG. 5C) and its linker region phosphorylation is not affected by DHHC7 (FIG. 8C).

Subcellular fractionation showed that DHHC7 knockout decreased the cytoplasmic localization of SMAD2 as well as the p-SMAD2(L3) located in the cytoplasm and nucleus (FIG. 8D). Interestingly, the ratio of p-SMAD2(L3) to SMAD2 was decreased even more in the nuclear fraction than in the cytoplasmic fraction (FIG. 8D). Similarly, immunofluorescence showed that in DHHC7-expressing cells, SMAD2 had more cytoplasmic localization and higher phosphorylation levels than DHHC7 knockout cells (FIG. 8E). All the data suggested that DHHC7-catalyzed SMAD2 palmitoylation promotes SMAD2 phosphorylation at the linker region.

### S-Palmitoylation Promotes the Binding of SMAD2 to STAT3 and SMAD4.

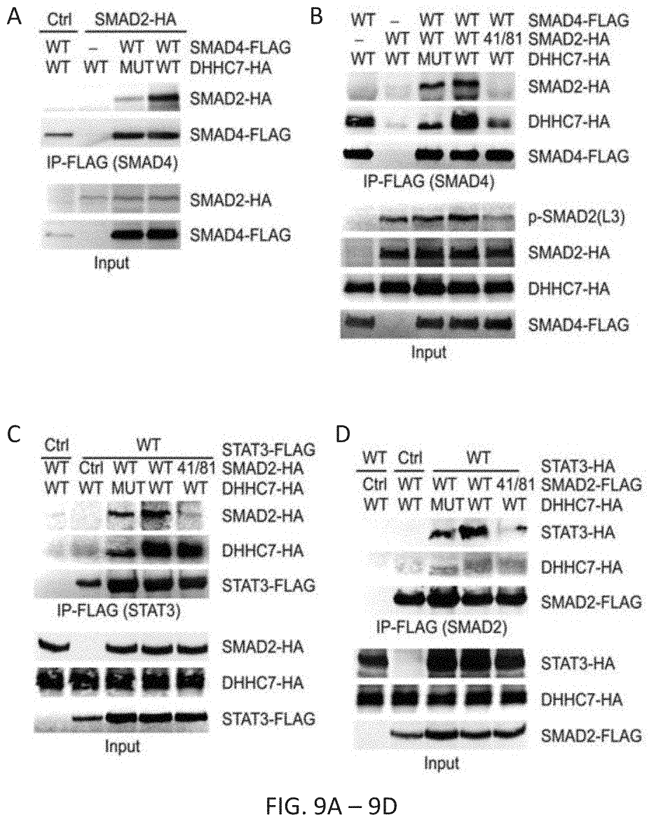

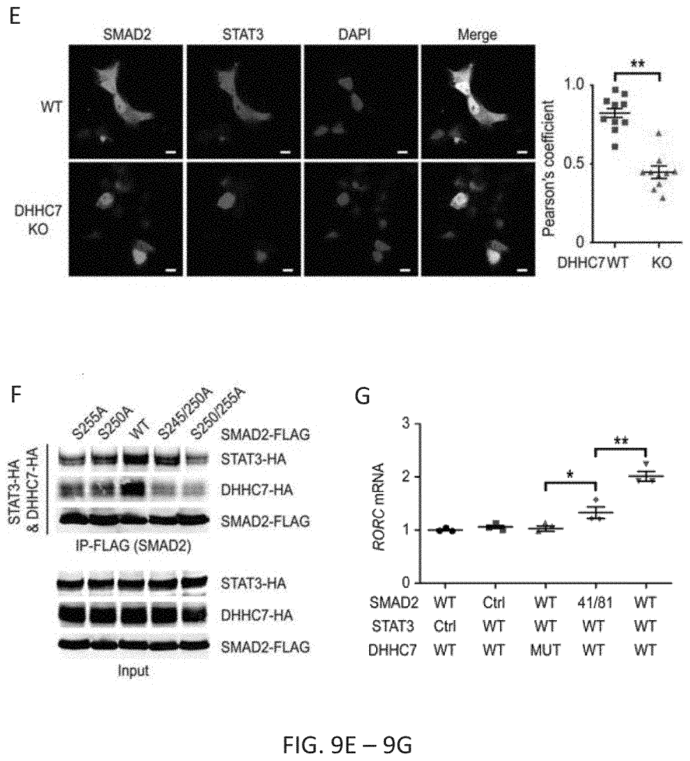

The inventors next investigated how DHHC7 promotes the function of SMAD2. As binding of SMAD4 is important for SMAD2's transcriptional activity, the inventors did the protein pull down assay and found that the interaction between SMAD2 and SMAD4 was increased by DHHC7-induced S-palmitoylation (FIG. 9A) and the binding of SMAD2 and SMAD4 was decreased by either catalytic inactive DHHC7 or SMAD2 Cys41/81Ser mutant (FIG. 9B).

Linker region Ser255 phosphorylated SMAD2 by ERK serves as a STAT3 co-activator in expressing RORγt and IL-17A. The inventors examined whether palmitoylation could facilitate SMAD2-STAT3 association. DHHC7 induced the formation of SMAD2/STAT3 complex while catalytic inactive DHHC7 or SMAD2 Cys41/81Ser mutant decreased the complex formation (FIGS. 9C-9D). Similarly, immunofluorescence showed that in DHHC7-expressing cells, SMAD2 had more cytoplasmic and nuclear colocalizations with STAT3 than DHHC7 knockout cells, which suggested DHHC7 promoted the interaction between SMAD2 and STAT3 (FIG. 9E). As there are three phosphorylation sites at linker region (Ser245/250/255) for SMAD2, the inventors checked which site is the key site for the formation of the SMAD2/STAT3 complex by mutagenesis. The inventors found Ser255Ala mutant of SMAD2 significantly decreased its affinity with the STAT3 (FIG. 9F). The inventors finally evaluated the RORC expression by real time PCR and found that WT SMAD2 promotes the RORC expression compared to the Cys41/81Ser mutant SMAD2 associated with STAT3 (FIG. 9G). These data provided additional support that S-palmitoylation of SMAD2 facilitates the binding with SMAD4 and STAT3 by promoting SMAD2 linker region phosphorylation.

### The S-Palmitoylation Cycle of SMAD2 Promotes T_{H}17 Cell Differentiation.

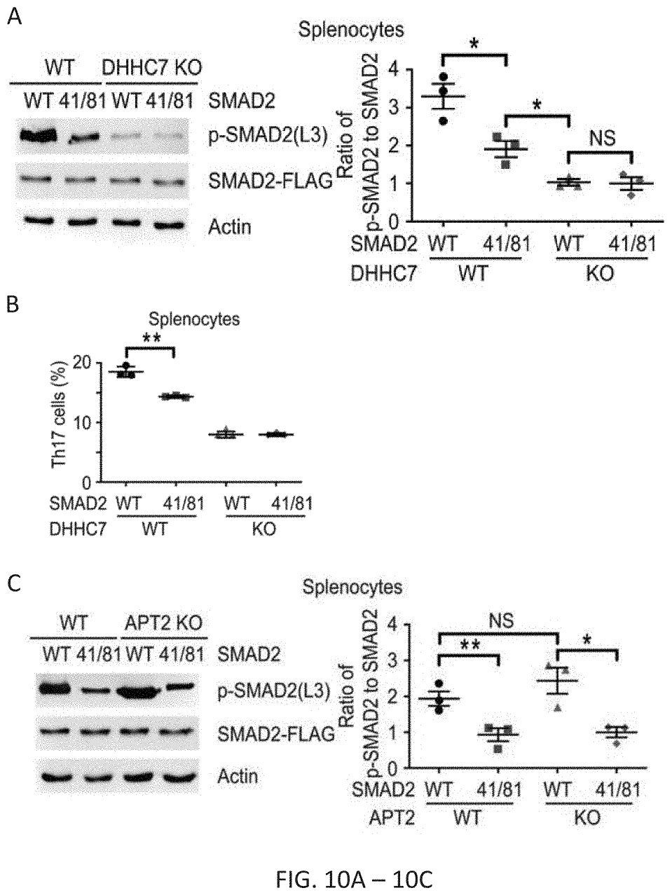

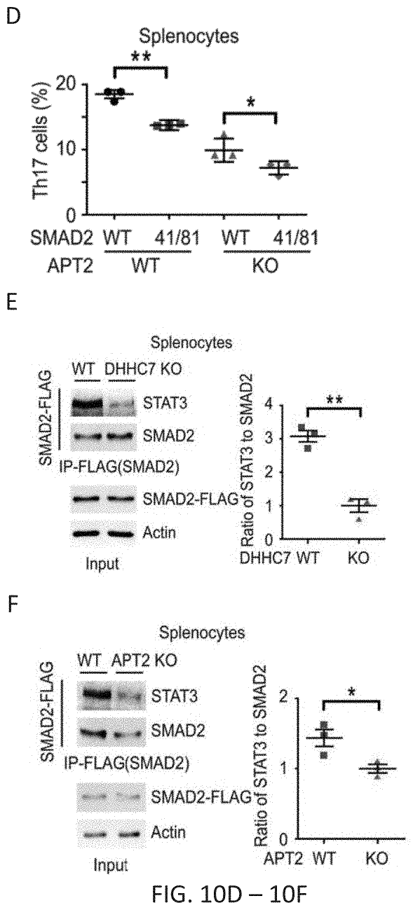

Given that SMAD2 and its association with STAT3 play a key role in the differentiation of naive T cells into T_{H}17 cells, the inventors examined if the SMAD2 S-palmitoylation can regulate the production of T_{H}17 cells in mouse splenocytes. The data in Example 3 above shows that the palmitoylation-depalmitoylation cycle of STAT3 is important for STAT3 activity and T_{H}17 cell differentiation. The inventors thus wondered a similar palmitoylation-depalmitoylation cycle also regulate SMAD2 activity and contribute to T_{H}17 differentiation. Compared with WT SMAD2, C41/81S SMAD2 in splenocytes showed decreased p-SMAD2(L3) (FIG. 10A). Zdhhc7 knockout reduced p-SMAD2(L3) for both WT and C41/81S SMAD2 (FIG. 10A). The fact that Zdhhc7 knockout also decreased p-SMAD2(L3) C41/81S suggests that DHHC7 may have other substrates which may contribute to SMAD2 phosphorylation in the linker region. Consistent with the p-SMAD2(L3) level, the T_{H}17 cell differentiation level was inhibited by either C41/81S mutant or Zdhhc7 knockout (FIG. 10B). Zdhhc7 knockout had a weak effect on SMAD2 C41/81S mutant likely because DHHC7 also regulate STAT3, another important factor for T_{H}17 differentiation as shown previously. APT2 knockout slightly increased p-SMAD2(L3) in splenocytes compared with WT APT2 but the difference was not statistically significant (FIG. 10C). Interestingly, different from p-SMAD2(L3) level, APT2 knockout inhibited the T_{H}17 cell differentiation in splenocytes overexpressing WT or C41/81S mutant SMAD2 (FIG. 10D). The inventors next evaluated the formation of SMAD2/STAT3 complex. Zdhhc7 knockout decreased the binding between SMAD2 and STAT3 (FIG. 10E). Consistent with the ideas that the palmitoylation-depalmitoylation cycle is important, APT2 knockout also decreased the SMAD2-STAT3 binding, which is also consistent with the T_{H}17 cell differentiation result (FIG. 10F). The above data suggests that the palmitoylation-depalmitoylation cycle catalyzed by DHHC7 and APT2 plays a significant role in promoting SMAD2-STAT3 binding and T_{H}17 differentiation.

### Inhibition of SMAD2 S-Palmitoylation Attenuates Th17 Cell Differentiation and Inflammation in Multiple Sclerosis Mice Models.

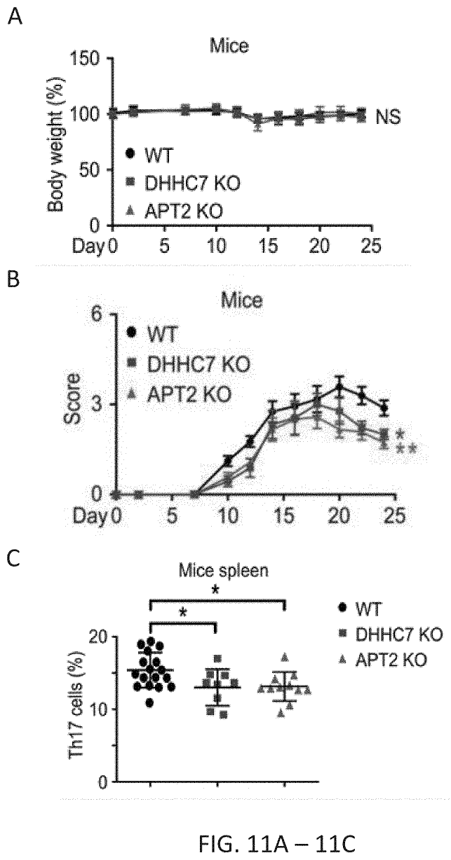

The proinflammatory role of T_{H}17 is well known to contribute to the pathogenesis in multiple sclerosis. To further test that targeting the palmitoylation-depalmitoylation cycle of SMAD2 would promote T_{H}17 and aggravate inflammation, the inventors studied the effect of DHHC7 and APT2 knockout in Myelin Oligodendrocyte Glycoprotein (MOG35-55) induced Experimental Autoimmune Encephalomyelitis (EAE) mice (a classical experimental model for MS). After the administration of MOG35-55 and pertussis toxin to induce EAE, the body weight had no significant reduction in WT, DHHC7 knockout, or APT2 knockout mice (FIG. 11A). Subsequently, the inventors scored the clinical symptoms of mice and found that both DHHC7 and APT2 knockout mice had a lower clinical score compared to the WT mice (FIG. 11B). Moreover, consistent with in vitro data (FIGS. 9C-9D), both DHHC7 and APT2 knockout decreased the T_{H}17 differentiation in mice spleen respectively (FIG. 11C). These findings indicate that S-palmitoylation of SMAD2 is effective target in preventing the inflammation of MS mice.

Regulated SMADs (R-SMADs), as direct substrates of the TGFβ receptor, undergoes nuclear-cytoplasm shuttling and plays a critical role in TGFβ signaling. Many post-translational modifications are involved in this process. For example, COOH-terminal tail phosphorylation is critical for R-SMADs activation and linker region phosphorylation is essential for complex formation with the functional DNA-binding transcription factor. Although SMAD2 and SMAD3 share many common functions, they have opposite roles in T cell differentiation with SMAD2 promoting T_{H}17 while SMAD3 promote Treg. How cells control these two similar R-SMADs to favor one or the other T cell differentiation process is still unclear. In this disclosure, the inventors found that SMAD2, not SMAD3, could be palmitoylated and the palmitoylation-depalmitoylation cycle facilitate the nuclear-cytoplasm shuttling and its association with Co-SMAD (SMAD4) and STAT3. This study demonstrates that the SMAD2 palmitoylation promotes its phosphorylation at the linker region, but in order to be active in T_{H}17 differentiation, it had to bind with STAT3 and depalmitoylated by APT2 to translocate to the nucleus. The present results showed that the S-palmitoylation cycle drives naive T cells differentiation to Th17 cells rather than Treg cells and promoted the inflammation in multiple sclerosis mouse model.

Intracellular dynamics of palmitoylation are regulated by a family of 23 palmitoyltransferases, known as DHHCs, that are defined by the presence of a conserved Asp-His-His-Cys sequence motif. Most DHHCs enzymes localize to cellular membranes, including those of the endoplasmic reticulum (ER), Golgi apparatus and plasma membrane. S-Palmitoylation is removed by acyl-protein thioesterases (APT1, APT2, and ABHD family members). With the screening of all the DHHCs and APTs, the inventors found that DHHC7 and APT2 catalyze SMAD2 palmitoylation and depalmitoylation, respectively, and promote T_{H}17 cell differentiation. However, the mechanistic detail of how DHHC7 and APT2-catalyzed SMAD2 palmitoylation-depalmitoylation cycle regulates SMAD2 phosphorylation and biological function is different from that of STAT3. DHHC7 palmitoylates STAT3 at N-terminal (Cys108) and promotes the membrane recruitment and phosphorylation at C-terminal (Tyr705). APT2 has higher affinity to the phosphorylated STAT3 (p-STAT3) and depalmitoylates it to allow its translocation to the nucleus. Here, the inventors found that DHHC7 palmitoylates SMAD2 but does not promote the TGFbR-mediated phosphorylation of SMAD2 at the C-terminal. Instead, palmitoylation promotes SMAD2 phosphorylation at the linker region, which is catalyzed by ERK. For SMAD2 depalmitoylation, APT2 has no preference for the C-terminal phosphorylated SMAD2 (Ser465/Ser467), unlike its preference for phosphorylated STAT3. The inventors found that as a partner of SMAD2, STAT3 preferentially binds with the linker phosphorylated SMAD2 and translocate to the nucleus to promote the RORC and IL17A genes. The nuclear translocation of SMAD2-STAT3 complex is promoted by APT2-catalyzed depalmitoylation. The inventors confirmed the phosphorylation at Ser255 is the major phosphorylation site that promotes the binding between SMAD2 and STAT3. This represents a palmitoylation-depalmitoylation cycle, controlled by DHHC7 and APT2, to activate SMAD2-STAT3 complex to promote T_{H}17 differentiation.

Although SMAD2 and SMAD3 are highly homologous and share the same upstream signaling pathway and even many downstream signaling outcomes, they opposingly promote T_{H}17 and Treg cells, respectively, in the process of T cell differentiation. Although a previous report elucidated the important role of SMAD2 linker phosphorylation in promoting T_{H}17 differentiation, how naïve T cells control STAT3 interaction with p-SMAD2(L3) instead of unphosphorylated SMAD3 is an unaddressed question. This study suggests that DHHC7-catalyzed S-palmitoylation could be the mechanism that promotes T_{H}17 while suppressing Treg, as the data suggested that SMAD2, but not SMAD3, is palmitoylated by DHHC7. The inventors postulate that in naïve T cells with more active DHHC7, SMAD2 will be palmitoylated and phosphorylated on the linker region, binds to STAT3 and promote the T_{H}17 pathway. In contrast, when there is no DHHC7, SMAD3 will be in complex with STAT3 and thus Treg differentiation will be favored.

The TGFβ-induced SMAD2 acts as an important transcriptional factor in pro-inflammatory T_{H}17 cells, characterized by the expression of IL-17. T_{H}17 cells are abundant in patients with MS and further increase during relapses. Thus, cells from the T_{H}17 axis represent a major target of MS therapeutics. Interestingly, no differential genes expression was observed for SMAD2, SMAD3 and SMAD4 on circulating CD4⁺ T cells from MS patients compared to healthy control. These suggested that post-translational regulation of SMAD2 could be very important for the pathogenesis of MS. After discovering that S-palmitoylation promotes SMAD2's role in T_{H}17 differentiation, the inventors further demonstrated that perturbation of SMAD2-STAT3 binding by targeting DHHC7 or APT2 inhibited T_{H}17 cell differentiation and protected mice in the EAE model for MS. These results suggest that targeting the S-palmitoylation of SMAD2/STAT3 is a promising MS therapeutic strategy. Given that T_{H}17 cells play an important role in a variety of autoimmune diseases (such as IBD, arthritis, type-I diabetes), as well as in cancers, graft-versus-host and infectious diseases, the DHHC7-APT2 controlled palmitoylation cycle is expected to provide new insights and treatment strategies for a variety of diseases.

### Example 5

2-BP, a Pan-DHHC Inhibitor, Decreases Cytokines mRNA Expression During the LPS Priming Step of Inflammasome Activation.

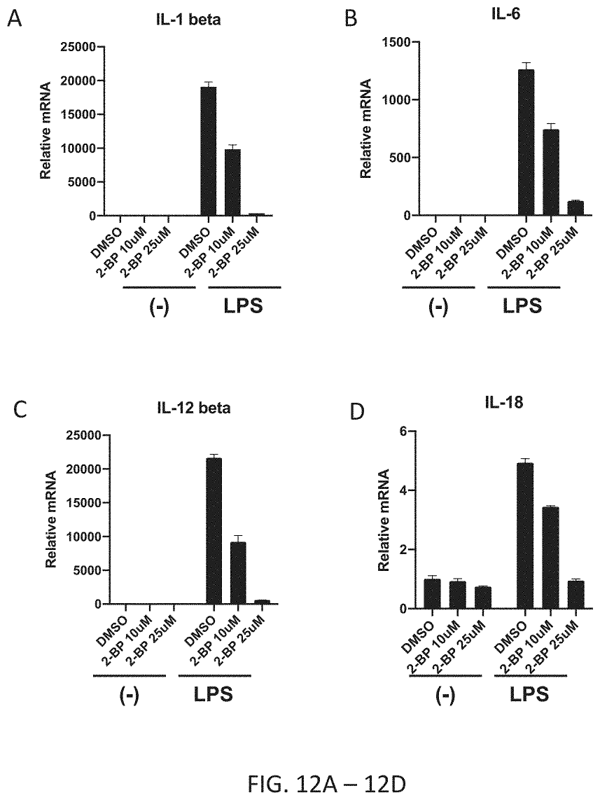

Bone marrow-derived macrophages (BMDM) were treated with 100 ng/mL lipopolysaccharide (LPS), which can prime BMDM for inflammasome activation. A small molecule that inhibits all the DHHC family of palmitoyltransferases (DHHCs), 2-bromopalmitate (2-BP), was added at 10 μM or 25 μM together with LPS. The cells were cultured with LPS and 2-BP for 6 hours and then the mRNA levels of several pro-inflammatory cytokines: IL-1 beta (FIG. 12A), IL-6 (FIG. 12B), IL-12 beta (FIG. 12C), and IL-18 (FIG. 12D), were measured by quantitative reverse transcription PCR (qRT-PCR). 2-BP can decrease the mRNA levels of all these cytokines in a concentration dependent manner, suggesting that inhibiting DHHCs can decrease the priming step of inflammasome activation.

### 2-BP Affects NLRP3-Mediated Inflammasome Activation.

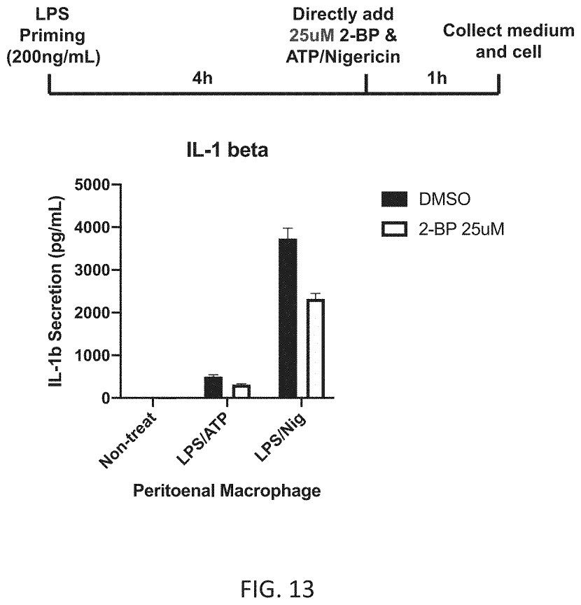

Peritoneal macrophage was first primed with LPS (200 ng/mL) in DMEM medium (without serum) for 4h, and then 2-BP (25 μM) and ATP (5 mM) or Nigericin (10 μM) were added to the cell culture and incubated for 1 h. ATP and nigericin are two reagents that are commonly used to activate the NLRP3 inflammasome. The activation of NLRP3 inflammasome was then monitored by measuring the level of IL-1 beta secreted to the medium (FIG. 13). The data support that inhibiting DHHCs with 2-BP can decrease NLRP3 inflammasome activation.

### DHHC7 Knockout Decreases IL-1b and IL-18 Secretion in Bone-Marrow-Derived Macrophage (BMDM) During Inflammasome Activation.

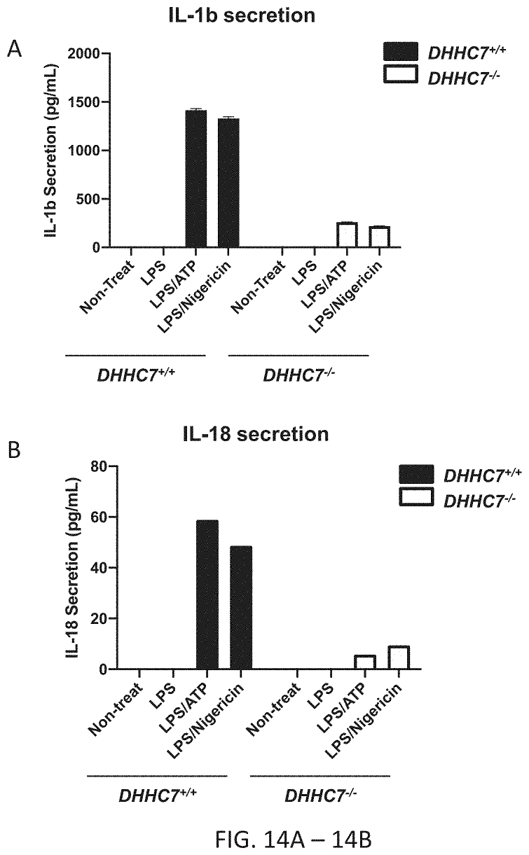

DHHC7 WT and knockout BMDM were primed with LPS (10 ng/mL) in DEME medium overnight. The next day, the medium was change to DMEM with LPS (10 ng/mL) and ATP (5 mM), or DMEM with LPS (10 ng/mL) and nigericin (10 μM) and incubated for 1 h to activate the NLRP3 inflammasome. The medium was then collected, and the secreted IL-1 beta (FIG. 14A) and IL-18 (FIG. 14B) were measured using ELISA kits. The results showed that DHHC7 knockout can significantly decrease NLRP3 inflammasome activity.

### APT2 Knockout has a Slight Effect on IL-1b Secretion in BMDM During Inflammasome Activation.

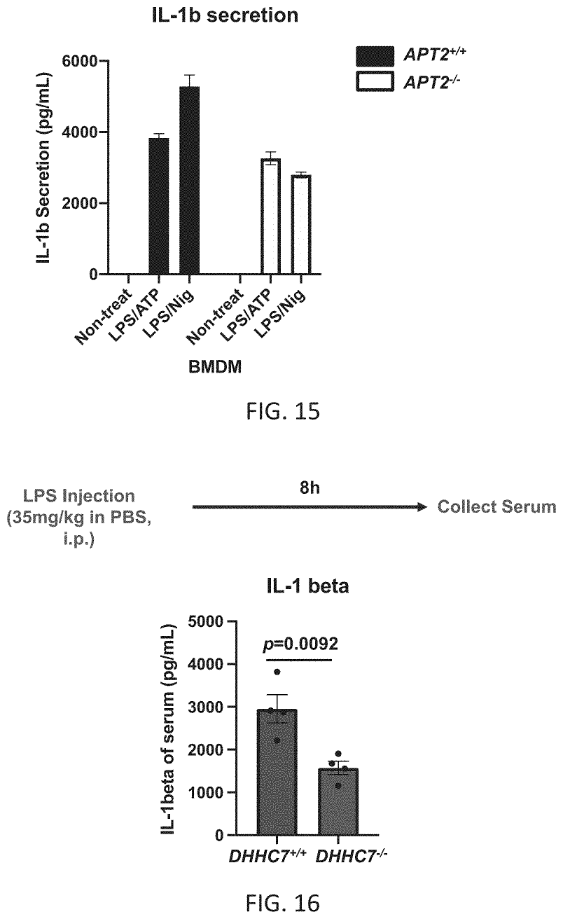

APT2 WT and knockout BMDM were primed with LPS (200 ng/mL) in DEME medium for 4 hours. Then, the medium was change to DMEM with LPS (200 ng/mL) and ATP (5 mM), or DMEM with LPS (10 ng/mL) and nigericin (10 μM) and incubated for 1 h to activate the NLRP3 inflammasome. The medium was then collected, and the secreted IL-1 beta was measured using an ELISA kit. The results showed that APT2 knockout can also decrease NLRP3 inflammasome activity, although less so compared to DHHC7 knockout (FIG. 15).

### DHHC7 Knockout Decreases Inflammasome Activation in Mice: LPS-Induced Endotoxic Shock.

Adult (>8 weeks old) B6.129P2(FVB) DHHC7 WT or knockout mice were injected intraperitoneally with LPS (35 mg/kg) in about 100 μL of sterile PBS buffer. 12 hours later, the mice will be euthanized, and the blood was collected for analysis of IL-1beta levels in the serum. DHHC7 knockout significantly decreased the amount of IL-1beta secreted to the serum, suggesting that DHHC7 knockout decreased inflammasome activation in the mice (FIG. 16).

### Lupus Nephrosis Mouse Model: APT2 Inhibitor ML349 Decreases Protein Concentration in the Mouse Urine.

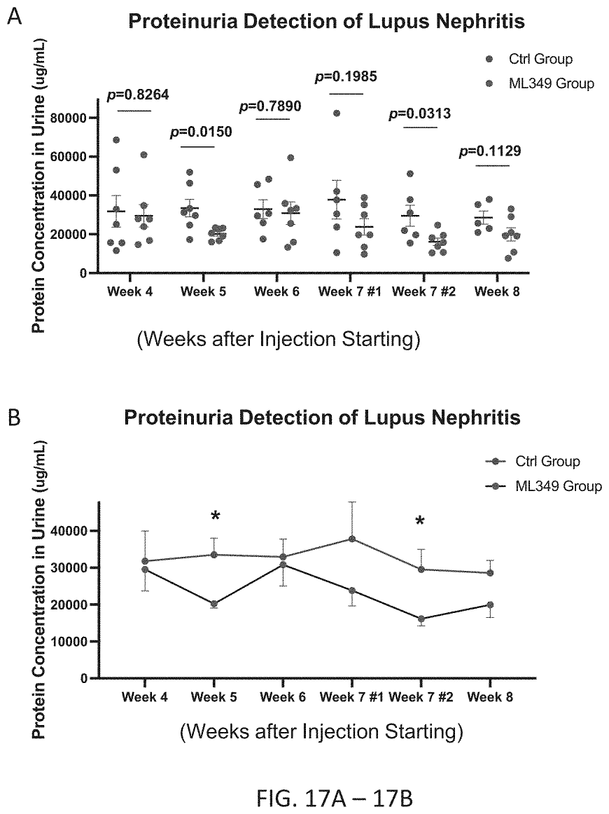

NZB/W F1 female mice at 25-week were administered via IP injection with either vehicle solution (DMSO+PBS) or APT2 inhibitor ML349 at 25 mg/Kg three times per week for 8 weeks. After 4 weeks of treatment, mice were evaluated for lupus incidence weekly through proteinuria analysis by collecting the urines and measuring the protein concentrations in the urine. The data showed that APT2 inhibitor ML349 can decrease the protein concentrations in the urine (FIGS. 17A-17B). This was the first time that the inventors did this experiment and after the experiment began, the inventors noticed that at 25 weeks, the disease phenotype was already very severe. It is expected that if the ML349 treatment is begun earlier, the effect might be more obvious and significant.

What is claimed is:

1. A method for treating an inflammatory disorder, comprising administering to a patient suffering from the disorder an effective amount of an inhibitor of an enzyme that regulates the S-palmitoylation of a pro-inflammatory transcription factor.

2. The method of claim 1, wherein the enzyme is Zinc finger DHHC-type palmitoyltransferase 7 (ZDHHC7) or Zinc finger DHHC-type palmitoyltransferase 3 (ZDHHC3).

3. The method of claim 1, wherein the enzyme is Lysophospholipase 2 (LYPLA2).

4. The method of claim 1, wherein the inhibitor of the enzyme is a nucleic acid inhibitor.

5. The method of claim 4, the nucleic acid inhibitor is selected from the group consisting of an antisense RNA, a small interfering RNA, a microRNA, an artificial microRNA, and a ribozyme.

6. The method of claim 1, wherein the inhibitor of the enzyme is a genome editing system.

7. The method of claim 6, wherein the genome editing system is selected from the group consisting of CRISPR/Cas system, Cre/Lox system, TALEN system, ZFNs system and homologous recombination.

8. The method of claim 7, wherein the CRISPR-mediated genome editing comprises introducing into the patient a first nucleic acid encoding a Cas9 nuclease, a second nucleic acid comprising a guide RNA (gRNA), wherein said gRNA is specific to the gene encoding the enzyme.

9. The method of claim 1, wherein the inhibitor of the enzyme is a small molecule inhibitor.

10. The method of claim 9, wherein the enzyme is LYPLA2, and the inhibitor is ML349 with the following chemical formula:

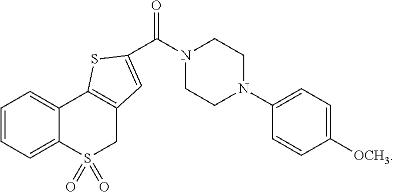 [SMILES: COc1ccc(N2CCN(C(=O)c3cc4c(s3)-c3ccccc3S(=O)(=O)C4)CC2)cc1]

11. The method of claim 9, wherein the enzyme is a Zinc finger DHHC-type palmitoyltransferase, and the inhibitor is selected from 2-bromopalmitic acid, cerulenin or tunicamycin.

12. The method of claim 1, wherein the disorder is an autoimmune disorder.

13. The method of claim 12, wherein the autoimmune disorder is selected from the group consisting of inflammatory bowel disease, multiple sclerosis, rheumatoid arthritis, lupus, graft versus host disease, type I diabetes, gout, asthma and psoriasis.

14. The method of claim 1, wherein the disorder is an endotoxic shock.
